(19) United States
(12) Patent Application Publication
(10) Pub. No.: US 2017/0007079 A1
(43) Pub. Date: Jan. 12, 2017

(54) Layered Fibrous Structures and Methods for Making Same

(71) Applicant: The Procter & Gamble Company, Cincinnati, OH (US)

(72) Inventors: Janese Christine O'Brien Stickney, Wyoming, OH (US); Brooke Marie Woods, Springfield Township, OH (US); Sharon Rae Antoun, Harrison, OH (US); David William Cabell, Cincinnati, OH (US)

(21) Appl. No.: 15/202,805

(22) Filed: Jul. 6, 2016

Related U.S. Application Data
(60) Provisional application No. 62/190,773, filed on Jul. 10, 2015.
(60) Provisional application No. 62/358,208, filed on Jul. 5, 2016.

(51) Int. Cl. A47K 10/16; B32B 5/02; A47K 10/32; A61L 15/28

(52) U.S. Cl. CPC A47K 10/16; A61L 15/28; B32B 5/02; A47K 10/32; B32B 2555/02

(57) ABSTRACT

Layered fibrous structures and more particularly to layered fibrous structures containing a plurality of hydroxyl polymer filaments present in at least one exterior layer of the layered fibrous structure at a level greater than the level of hydroxyl polymer filaments present in known layered fibrous structures and methods for making such layered fibrous structures are provided.

### FIELD OF THE INVENTION

The present invention relates to layered fibrous structures and more particularly to layered fibrous structures comprising a plurality of hydroxyl polymer filaments present in at least one exterior layer of the layered fibrous structure at a level greater than the level of hydroxyl polymer filaments present in known layered fibrous structures and methods for making such layered fibrous structures.

### BACKGROUND OF THE INVENTION

Layered fibrous structures comprising a plurality of hydroxyl polymer filaments, such as starch filaments, are known in the art. However, such known layered fibrous structures have limited the level of hydroxyl polymer filaments present in the layered fibrous structures' exterior layers to 3 gsm or less due to higher levels (greater than 3 and/or greater than 5 and/or greater than 6 and/or greater than 10 gsm) of hydroxyl polymer filaments present in an exterior layer of a layered fibrous structure exhibit wet feel negatives, such as a slimy and/or gooey feel during use by a consumer, especially when the hydroxyl polymer filaments come in contact with moisture, such as in bowel movement and/or urine. Formulators believe that this is due to the materials present in the hydroxyl polymer filaments, for example the type of hydroxyl polymer and/or the effectiveness of the crosslinking of the hydroxyl polymer, which relates at least partially to the level of base such as triethanolamine present in the crosslinking agent used to make the hydroxyl polymer fibrous elements. For example, crosslinking agents used to make known layered fibrous structures comprising hydroxyl polymer filaments in at least one of the exterior layers of the layered fibrous structure comprised 2% by weight or more of base.

Prior Art FIG. 1A illustrates an example of a known layered fibrous structure 10 comprising an exterior layer comprising a plurality of hydroxyl polymer filaments, such as starch filaments, present at a level of 3 gsm or less. The layered fibrous structure may also comprise a pulp fiber layer 14.

Prior Art FIG. 1B illustrates another example of a known layered fibrous structure 10 comprising two exterior layers 12 comprising a plurality of hydroxyl polymer filaments, such as starch filaments, present at a level of 3 gsm or less, and an additional layer 14 sandwiched between the two exterior layers 12. The additional layer 14 may comprise filaments, fibers, and mixtures thereof. In one example, the additional layer 14 comprises pulp fibers. In another example, the additional layer 14 comprises a mixture of filaments and pulp fibers.

Prior Art FIG. 1C illustrates yet another example of a known layered fibrous structure 10 which comprises a first ply 16 and a second ply 18, which may be glued together to form a multi-ply layered fibrous structure. The first ply 16 comprises an exterior layer 12 comprising a plurality of hydroxyl polymer filaments, such as starch filaments, present at a level of 3 gsm or less, an additional layer 14 comprising pulp fibers, and a third layer 20 comprising a plurality of hydroxyl polymer filaments, such as starch filaments, present at a level of greater than 3 gsm, for example 16 gsm. The second ply 18, in this case, has the same composition as the first ply 16.

However, the level of hydroxyl polymer filaments present in a layered fibrous structure's exterior layer influences the dry surface and/or softness properties of the layered fibrous structure.

One problem with known layered fibrous structures comprising a plurality of hydroxyl polymer filaments in at least one exterior layer of the layered fibrous structure is that the known layered fibrous structures limited the level of hydroxyl polymer filaments to about 2 to 3 gsm, and/or less than 6 gsm and/or less than 10 gsm at an extreme. Such low levels of hydroxyl polymer filaments in a layered fibrous structure's exterior layer resulted in such layered fibrous structures exhibiting less softness than desired by consumers of such layered fibrous structures.

Accordingly, there is a need for layered fibrous structures comprising hydroxyl polymer filaments present in at least one exterior layer of the layered fibrous structure at a level of greater than 10 gsm, and methods for making such layered fibrous structures.

### SUMMARY OF THE INVENTION

The present invention fulfills the needs described above by providing layered fibrous structures comprising a plurality of hydroxyl polymer filaments present in at least one exterior layer of the layered fibrous structures present at a level that provides improved softness compared to known layered fibrous structures comprising lower levels of hydroxyl polymer filaments in their exterior layers, and methods for making same.

One solution to the problem identified above is to make layered fibrous structures comprising a plurality of hydroxyl polymer filaments present in at least one exterior layer of the layered fibrous structure at greater than 10 gsm such that the layered fibrous structure exhibits greater softness than known layered fibrous structures comprising lower levels (2 to 3 gsm for example) of hydroxyl polymer filaments in their exterior layers. In one example, the improved ability to increase the level of hydroxyl polymer filaments in the exterior layers of the layered fibrous structures of the present invention is attributable to features of the polymer melt composition as described herein, for example the type of hydroxyl polymer and/or the effectiveness of the crosslinking of the hydroxyl polymer, which relates at least partially to the level of base such as triethanolamine present in the crosslinking agent used to make the hydroxyl polymer fibrous elements, for example less than 2% and/or less than 1.8% and/or less than 1.5% and/or less than 1.25% and/or about 0% and/or about 0.25% and/or about 0.5% by weight, to produce the hydroxyl polymer fibrous elements.

In one example of the present invention, a layered fibrous structure comprising a plurality of hydroxyl polymer filaments present in at least one exterior layer of the layered fibrous structure at a level of greater than 10 and/or greater than 12 and/or greater than 14 and/or greater than 16 and/or at least 18 and/or less than 40 and/or less than 35 and/or less than 30 and/or less than 25 gsm is provided.

In another example of the present invention, a layered fibrous structure comprising a first outer layer comprising a plurality of hydroxyl polymer filaments present in the first outer layer at a basis weight of greater than 10 gsm is provided.

In still yet another example of the present invention, a method for making a layered fibrous structure according to the present invention, the method comprising the steps of:
a. spinning a polymer melt composition comprising a hydroxyl polymer, a crosslinking agent, and a surfactant into a plurality of hydroxyl polymer filaments; and
b. collecting the hydroxyl polymer filaments on a collection device such that a layered fibrous structure comprising hydroxyl polymer filaments that are present in an exterior layer of the layered fibrous structure at a level of greater than 10 and/or greater than 12 and/or greater than 14 and/or greater than 16 and/or at least 18 and/or less than 40 and/or less than 35 and/or less than 30 and/or less than 25 gsm is formed is provided.

The present invention provides novel layered fibrous structures that comprise a higher level of hydroxyl polymer filaments in at least one exterior layer of the layered fibrous structure compared to known layered fibrous structures comprising hydroxyl polymer filaments in their exterior layers, and methods for making such layered fibrous structures.

### BRIEF DESCRIPTION OF THE DRAWINGS

FIG. 1A is a schematic representation of an example of a prior art layered fibrous structure;

FIG. 1B is a schematic representation of another example of a prior art layered fibrous structure;

FIG. 1C is a schematic representation of another example of a prior art layered fibrous structure;

FIG. 2 is a schematic representation of an example of a layered fibrous structure according to the present invention;

FIG. 3 is a schematic representation of another example of a layered fibrous structure according to the present invention;

FIG. 4 is a schematic representation of another example of a layered fibrous structure according to the present invention;

FIG. 5 is a schematic representation of one example of a method for making a layered fibrous structure according to the present invention;

FIG. 6 is a schematic representation of one example of a portion of layered fibrous structure making process according to the present invention; and

FIG. 7 is a schematic representation of an example of a meltblow die in accordance with the present invention.

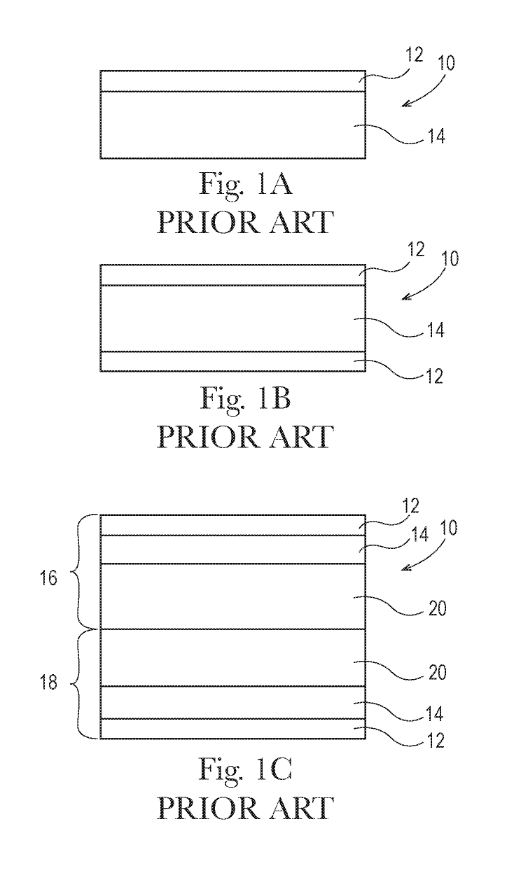

### DETAILED DESCRIPTION OF THE INVENTION

### Definitions

“Fibrous element” as used herein means an elongate particulate having a length greatly exceeding its average diameter, i.e. a length to average diameter ratio of at least about 10. A fibrous element may be a filament or a fiber. In one example, the fibrous element is a single fibrous element rather than a yarn comprising a plurality of fibrous elements.

The fibrous elements of the present invention may be spun from polymer melt compositions via suitable spinning operations, such as meltblowing and/or spunbonding and/or they may be obtained from natural sources such as vegetative sources, for example trees.

The fibrous elements of the present invention may be monocomponent and/or multicomponent. For example, the fibrous elements may comprise bicomponent fibers and/or filaments. The bicomponent fibers and/or filaments may be in any form, such as side-by-side, core and sheath, islands-in-the-sea and the like.

“Filament” as used herein means an elongate particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers. Non-limiting examples of filaments include meltblown and/or spunbond filaments. Non-limiting examples of polymers that can be spun into filaments include natural polymers, such as starch, starch derivatives, cellulose, such as rayon and/or lyocell, and cellulose derivatives, hemicellulose, hemicellulose derivatives, and synthetic polymers including, but not limited to polyvinyl alcohol, thermoplastic polymer, such as polyesters, nylons, polyolefins such as polypropylene filaments, polyethylene filaments, and biodegradable thermoplastic fibers such as polylactic acid filaments, polyhydroxyalkanoate filaments, polyesteramide filaments and polycaprolactone filaments.

“Fiber” as used herein means an elongate particulate as described above that exhibits a length of less than 5.08 cm (2 in.) and/or less than 3.81 cm (1.5 in.) and/or less than 2.54 cm (1 in.).

Fibers are typically considered discontinuous in nature. Non-limiting examples of fibers include pulp fibers, such as wood pulp fibers, and synthetic staple fibers such as polypropylene, polyethylene, polyester, copolymers thereof, rayon, glass fibers and polyvinyl alcohol fibers.

Staple fibers may be produced by spinning a filament tow and then cutting the tow into segments of less than 5.08 cm (2 in.) thus producing fibers.

In one example of the present invention, a fiber may be a naturally occurring fiber, which means it is obtained from a naturally occurring source, such as a vegetative source, for example a tree and/or plant, such as trichomes. Such fibers are typically used in papermaking and are oftentimes referred to as papermaking fibers. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to layered fibrous structures made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. Also applicable to the present invention are fibers derived from recycled paper, which may contain any or all of the above categories of fibers as well as other non-fibrous polymers such as fillers, softening agents, wet and dry strength agents, and adhesives used to facilitate the original papermaking.

In addition to the various wood pulp fibers, other cellulosic fibers such as cotton linters, rayon, lyocell, and bagasse fibers can be used in the layered fibrous structures of the present invention.

“Layered fibrous structure” as used herein means a structure that comprises one or more fibrous elements. In one example, a layered fibrous structure according to the present invention means an association of fibrous elements that together form a structure capable of performing a function. In another example of the present invention, a layered fibrous structure comprises a plurality of inter-entangled fibrous elements, for example filaments.

“Sanitary tissue product” as used herein means a soft, relatively low density layered fibrous structure useful as a wiping implement for post-urinary and post-bowel movement cleaning (toilet tissue), for otorhinolaryngological discharges (facial tissue), multi-functional absorbent and cleaning uses (absorbent towels) and wipes, such as wet and dry wipes. The sanitary tissue product may be convolutedly wound upon itself about a core or without a core to form a sanitary tissue product roll or may be in the form of discrete sheets.

In one example, the sanitary tissue product of the present invention comprises one or more layered fibrous structures according to the present invention.

The sanitary tissue products and/or layered fibrous structures of the present invention may exhibit a basis weight between about 1 g/m² to about 5000 g/m² and/or from about 10 g/m² to about 500 g/m² and/or from about 10 g/m² to about 300 g/m² and/or from about 10 g/m² to about 120 g/m² and/or from about 15 g/m² to about 110 g/m² and/or from about 20 g/m² to about 100 g/m² and/or from about 30 to 90 g/m² as determined by the Basis Weight Test Method described herein. In addition, the sanitary tissue product of the present invention may exhibit a basis weight between about 40 g/m² to about 120 g/m² and/or from about 50 g/m² to about 110 g/m² and/or from about 55 g/m² to about 105 g/m² and/or from about 60 g/m² to 100 g/m² as determined by the Basis Weight Test Method described herein.

The sanitary tissue products of the present invention may exhibit a total dry tensile strength of greater than about 59 g/cm and/or from about 78 g/cm to about 394 g/cm and/or from about 98 g/cm to about 335 g/cm. In addition, the sanitary tissue product of the present invention may exhibit a total dry tensile strength of greater than about 196 g/cm and/or from about 196 g/cm to about 394 g/cm and/or from about 216 g/cm to about 335 g/cm and/or from about 236 g/cm to about 315 g/cm. In one example, the sanitary tissue product exhibits a total dry tensile strength of less than about 394 g/cm and/or less than about 335 g/cm.

The sanitary tissue products of the present invention may exhibit a density of less than 0.60 g/cm³ and/or less than 0.30 g/cm³ and/or less than 0.20 g/cm³ and/or less than 0.15 g/cm³ and/or less than 0.10 g/cm³ and/or less than 0.07 g/cm³ and/or less than 0.05 g/cm³ and/or from about 0.01 g/cm³ to about 0.20 g/cm³ and/or from about 0.02 g/cm³ to about 0.15 g/cm³ and/or from about 0.02 g/cm³ to about 0.10 g/cm³.

The sanitary tissue products of the present invention may be in the form of sanitary tissue product rolls. Such sanitary tissue product rolls may comprise a plurality of connected, but perforated sheets of layered fibrous structure, that are separably dispensable from adjacent sheets.

The sanitary tissue products of the present invention may comprise additives such as softening agents, temporary wet strength agents, permanent wet strength agents, bulk softening agents, lotions, silicones, wetting agents, latexes, patterned latexes and other types of additives suitable for inclusion in and/or on sanitary tissue products.

“Scrim” as used herein means a material that is used to overlay solid additives present on and/or within a nonwoven substrate of the layered fibrous structures of the present invention such that the solid additives are positioned between the scrim and a layer of the layered fibrous structure. In one example, the scrim covers the solid additives such that they are positioned between the scrim and a surface of the nonwoven substrate of the layered fibrous structure. In another example, the scrim is a minor component (for example less than 25% of the basis weight) relative to the nonwoven substrate of the basis weight of the layered fibrous structure. In one example, the scrim forms at least one exterior layer and/or exterior surface of a layered fibrous structure of the present invention.

“Hydroxyl polymer” as used herein includes any hydroxyl-containing polymer that can be incorporated into a filament of the present invention. In one example, the hydroxyl polymer of the present invention includes greater than 10% and/or greater than 20% and/or greater than 25% by weight hydroxyl moieties. In another example, the hydroxyl within the hydroxyl-containing polymer is not part of a larger functional group such as a carboxylic acid group.

“Non-thermoplastic” as used herein means, with respect to a material, such as a fibrous element as a whole and/or a polymer, such as a crosslinked polymer, within a fibrous element, that the fibrous element and/or polymer exhibits no melting point and/or softening point, which allows it to flow under pressure, in the absence of a plasticizer, such as water, glycerin, sorbitol, urea and the like.

“Non-cellulose-containing” as used herein means that less than 5% and/or less than 3% and/or less than 1% and/or less than 0.1% and/or 0% by weight of cellulose polymer, cellulose derivative polymer and/or cellulose copolymer is present in fibrous element. In one example, “non-cellulose-containing” means that less than 5% and/or less than 3% and/or less than 1% and/or less than 0.1% and/or 0% by weight of cellulose polymer is present in fibrous element.

“Crosslinking facilitator” and/or “crosslinking facilitator function” as used herein means any material that is capable of activating a crosslinking agent thereby transforming the crosslinking agent from its unactivated state to its activated state.

“Fast wetting surfactant” and/or “fast wetting surfactant component” and/or “fast wetting surfactant function” as used herein means a surfactant and/or surfactant component, such as an ion from a fast wetting surfactant, for example a sulfosuccinate diester ion (anion), that exhibits a Critical Micelle Concentration (CMC) of greater 0.15% by weight and/or at least 0.25% and/or at least 0.50% and/or at least 0.75% and/or at least 1.0% and/or at least 1.25% and/or at least 1.4% and/or less than 10.0% and/or less than 7.0% and/or less than 4.0% and/or less than 3.0% and/or less than 2.0% by weight.

“Polymer melt composition” or “Polysaccharide melt composition” as used herein means a composition comprising water and a melt processed polymer, such as a melt processed fibrous element-forming polymer, for example a melt processed hydroxyl polymer, such as a melt processed polysaccharide.

“Melt processed fibrous element-forming polymer” as used herein means any polymer, which by influence of elevated temperatures, pressure and/or external plasticizers may be softened to such a degree that it can be brought into a flowable state, and in this condition may be shaped as desired.

“Melt processed hydroxyl polymer” as used herein means any polymer that contains greater than 10% and/or greater than 20% and/or greater than 25% by weight hydroxyl groups and that has been melt processed, with or without the aid of an external plasticizer. More generally, melt processed hydroxyl polymers include polymers, which by the influence of elevated temperatures, pressure and/or external plasticizers may be softened to such a degree that they can be brought into a flowable state, and in this condition may be shaped as desired.

“Blend” as used herein means that two or more materials, such as a fibrous element-forming polymer, for example a hydroxyl polymer and a polyacrylamide are in contact with each other, such as mixed together homogeneously or non-homogeneously, within a filament. In other words, a filament formed from one material, but having an exterior coating of another material is not a blend of materials for purposes of the present invention. However, a fibrous element formed from two different materials is a blend of materials for purposes of the present invention even if the fibrous element further comprises an exterior coating of a material.

“Associate,” “Associated,” “Association,” and/or “Associating” as used herein with respect to fibrous elements means combining, either in direct contact or in indirect contact, fibrous elements such that a layered fibrous structure is formed. In one example, the associated fibrous elements may be bonded together for example by adhesives and/or thermal bonds. In another example, the fibrous elements may be associated with one another by being deposited onto the same layered fibrous structure making belt.

“Average Diameter” as used herein, with respect to a fibrous element, is measured according to the Average Diameter Test Method described herein. In one example, a fibrous element of the present invention exhibits an average diameter of less than 50 μm and/or less than 25 μm and/or less than 20 μm and/or less than 15 μm and/or less than 10 μm and/or less than 6 μm and/or greater than 1 μm and/or greater than 3 μm.

“Basis Weight” as used herein is the weight per unit area of a sample reported in lbs/3000 ft² or g/m² as determined by the Basis Weight Test Method described herein.

“Machine Direction” or “MD” as used herein means the direction parallel to the flow of the layered fibrous structure through a layered fibrous structure making machine and/or sanitary tissue product manufacturing equipment. Typically, the MD is substantially perpendicular to any perforations present in the layered fibrous structure

“Cross Machine Direction” or “CD” as used herein means the direction perpendicular to the machine direction in the same plane of the layered fibrous structure and/or sanitary tissue product comprising the layered fibrous structure.

“Ply” or “Plies” as used herein means an individual layered fibrous structure optionally to be disposed in a substantially contiguous, face-to-face relationship with other plies, forming a multiple ply layered fibrous structure. It is also contemplated that a single layered fibrous structure can effectively form two “plies” or multiple “plies”, for example, by being folded on itself.

As used herein, the articles “a” and “an” when used herein, for example, “an anionic surfactant” or “a fiber” is understood to mean one or more of the material that is claimed or described.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Layered Fibrous Structures

In one example, the layered fibrous structures of the present invention comprise a plurality of fibrous elements, for example hydroxyl polymer filaments comprising a hydroxyl polymer such as a crosslinked hydroxyl polymer. In another example, the layered fibrous structures may comprise starch and/or starch derivative filaments. The starch filaments may further comprise polyvinyl alcohol and/or other polymers.

As shown in FIG. 2, an example of a layered fibrous structure 10 according to the present invention, which may be a single ply fibrous structure, comprises two exterior layers 12 comprising a plurality of hydroxyl polymer filaments, such as starch filaments, present at a level of greater than 10 and/or greater than 12 and/or greater than 14 and/or greater than 16 and/or at least 18 and/or less than 40 and/or less than 35 and/or less than 30 and/or less than 25 gsm. An additional layer 14 is sandwiched between the two exterior layers 12 and may comprise a plurality of fibers, such as pulp fibers. The fibers may be present in the additional layer 14 at a level of greater than 6 and/or greater than 8 and/or greater than greater than 10 and/or greater than 12 and/or greater than 14 and/or greater than 16 and/or at least 18 and/or less than 40 and/or less than 35 and/or less than 30 and/or less than 25 gsm.

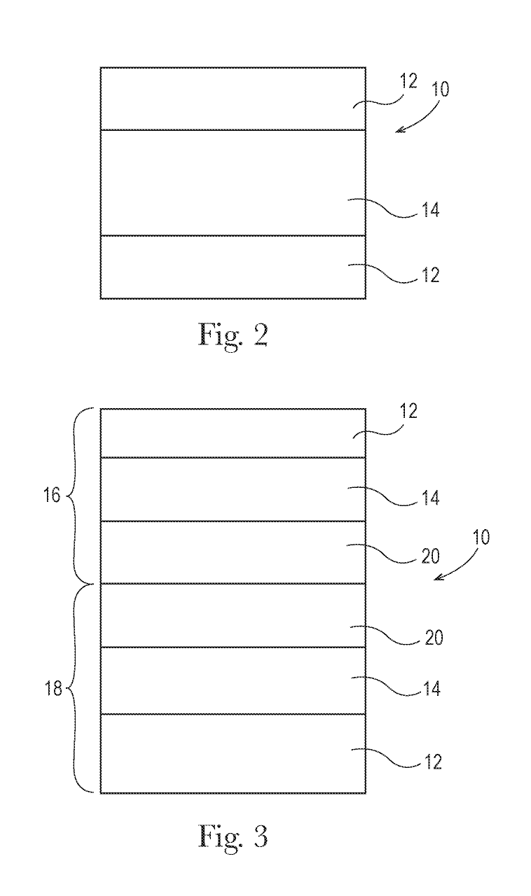

As shown in FIG. 3, another example of a layered fibrous structure 10 according to the present invention comprises a first ply 16 and a second ply 18, which may be glued together to form a multi-ply layered fibrous structure. The first ply 16 comprises an exterior layer 12 comprising a plurality of hydroxyl polymer filaments, such as starch filaments, present at a level of greater than 6 and/or greater than 8 and/or greater than 10 and/or greater than 12 and/or greater than 14 and/or greater than 16 and/or at least 18 and/or less than 40 and/or less than 35 and/or less than 30 and/or less than 25 gsm, an additional layer 14 comprising fibers, such as pulp fibers, present at a level of greater than 6 and/or greater than 8 and/or greater than greater than 10 and/or greater than 12 and/or greater than 14 and/or greater than 16 and/or at least 18 and/or less than 40 and/or less than 35 and/or less than 30 and/or less than 25 gsm, and a third layer 20 comprising a plurality of hydroxyl polymer filaments, such as starch filaments, present at a level of greater than 12 and/or greater than 14 and/or greater than 16 and/or at least 18 and/or less than 40 and/or less than 35 and/or less than 30 and/or less than 25 gsm. The second ply 18 and first ply 16 may have different compositions or the same composition. In this case, the second ply 18 has a different composition from the first ply 16. The second ply 18 comprises an exterior layer 12 comprising a plurality of hydroxyl polymer filaments, such as starch filaments, present at a level of greater than 12 and/or greater than 14 and/or greater than 16 and/or at least 18 and/or less than 40 and/or less than 35 and/or less than 30 and/or less than 25 gsm, an additional layer 14 comprising fibers, such as pulp fibers, present at a level of greater than 6 and/or greater than 8 and/or greater than greater than 10 and/or greater than 12 and/or greater than 14 and/or greater than 16 and/or at least 18 and/or less than 40 and/or less than 35 and/or less than 30 and/or less than 25 gsm, and a third layer 20 comprising a plurality of hydroxyl polymer filaments, such as starch filaments, present at a level of greater than 6 and/or greater than 8 and/or greater than 10 and/or greater than 12 and/or greater than 14 and/or greater than 16 and/or at least 18 and/or less than 40 and/or less than 35 and/or less than 30 and/or less than 25 gsm.

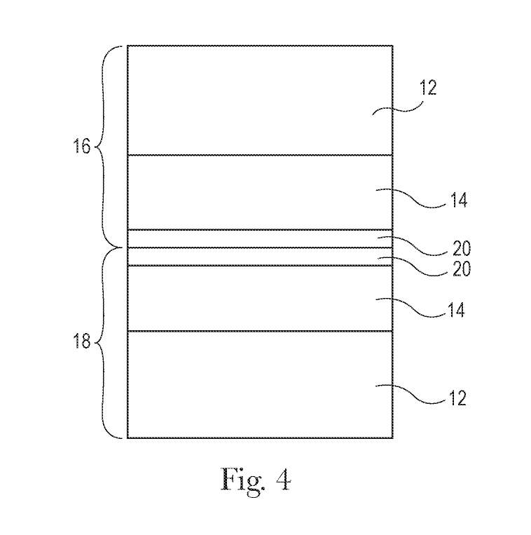

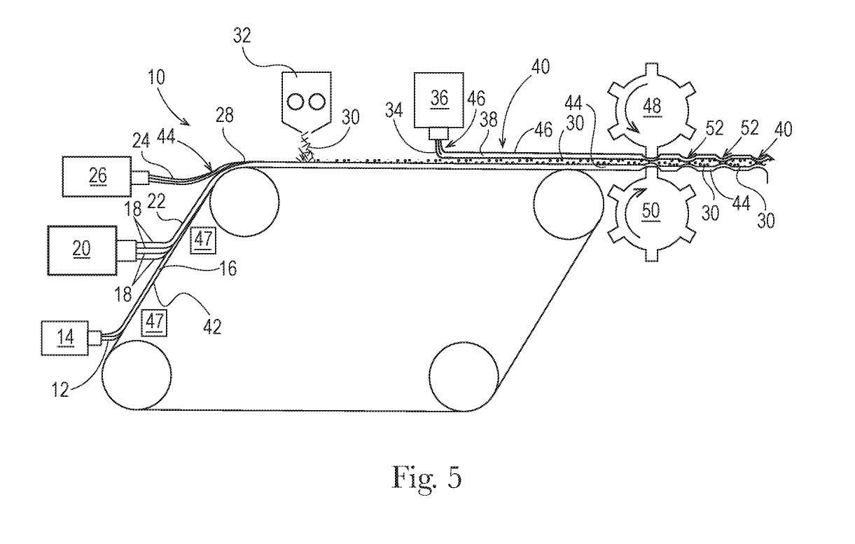

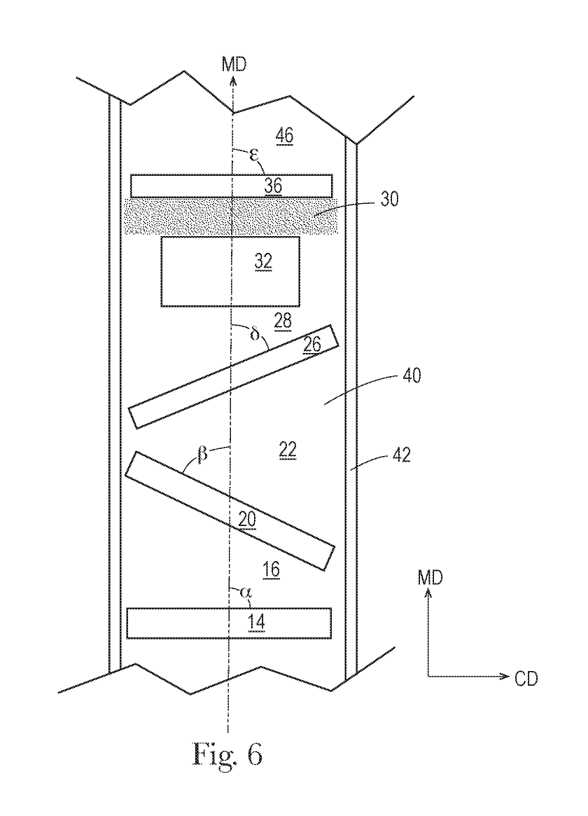

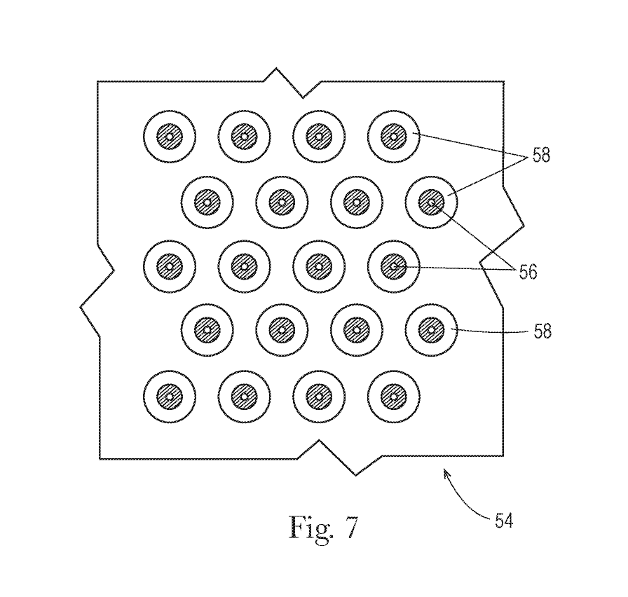

FIG. 4 shows yet another example of a layered fibrous structure 10 of the present invention. This layered fibrous structure 10 comprises a first ply 16 and a second ply 18, which may be glued together to form a multi-ply layered fibrous structure. The first ply 16 comprises an exterior layer 12 comprising a plurality of hydroxyl polymer filaments, such as starch filaments, present at a level of greater than 10 and/or greater than 12 and/or greater than 14 and/or greater than 16 and/or at least 18 and/or less than 40 and/or less than 35 and/or less than 30 and/or less than 25 gsm, an additional layer 14 comprising fibers, such as pulp fibers, present at a level of greater than 6 and/or greater than 8 and/or greater than greater than 10 and/or greater than 12 and/or greater than 14 and/or greater than 16 and/or at least 18 and/or less than 40 and/or less than 35 and/or less than 30 and/or less than 25 gsm, and a third layer 20 comprising a plurality of hydroxyl polymer filaments, such as starch filaments, present at a level of greater than 1 and/or greater than 2 and/or less than 10 and/or less than 7 and/or less than 5 gsm. The second ply 18 and first ply 16 may have different compositions or the same composition. In this case, the second ply 18 has the same composition as the first ply 16.

The layered fibrous structures of the present invention may comprise a mixture of different fibrous elements for example a mixture of filaments, for example hydroxyl polymer filaments such as starch filaments, and fibers, for example cellulose fibers such as pulp fibers. The mixture of fibrous elements may comprise a commingled mixture of filaments and fibers and/or a layered association of filaments and fibers such that the layered fibrous structure exhibits novel surface and/or softness properties.

The layered fibrous structures of the present invention may be thermally bonded to bond the fibrous elements together to provide the layered fibrous structure with strength sufficient for its intended use, such as for use as bath tissue, and to control the pills, for example such that the layered fibrous structure exhibits a Dry Pill Area value of less than 5% and/or less than 4% and/or less than 3% and/or less than 2.5% as measured according to the Lint/Pilling Test Method described herein.

In one example, the layered fibrous structure comprises a layered fibrous structure comprising a least one exterior layer comprising a plurality of hydroxyl polymer filaments present at a level of greater than 10 and/or greater than 12 and/or greater than 14 and/or greater than 16 and/or greater than 18 and/or less than 40 and/or less than 35 and/or less than 30 and/or less than 25 gsm and another layer of solid additives, such as pulp fibers present at a level of greater than 6 and/or greater than 10 and/or greater than 12 and/or greater than 14 and/or greater than 16 and/or greater than 18 and/or less than 40 and/or less than 35 and/or less than 30 and/or less than 25 gsm and/or from about 6 to about 40 gsm and/or from about 10 to about 35 gsm and/or from about 12 to about 25 gsm, such that the layered fibrous structure exhibits novel surface and/or softness properties.

In one example, the layered fibrous structures of the present invention may comprise a plurality of fibrous elements, for example hydroxyl polymer filaments, such as at least one exterior surface comprising a plurality of hydroxyl polymer filaments, such that the layered fibrous structures exhibitnovel softness properties

In another example of the present invention, a layered fibrous structure comprising a plurality of hydroxyl polymer filaments that are present in an exterior layer and/or on an exterior surface of the layered fibrous structure at a level of greater than 10 and/or greater than 12 and/or greater than 14 and/or greater than 16 and/or at least 18 and/or less than 40 and/or less than 35 and/or less than 30 and/or less than 25 gsm, such that the layered fibrous structure exhibits novel surface and/or softness properties.

In another example, the layered fibrous structures of the present invention may comprise a plurality of fibrous elements, for example hydroxyl polymer filaments, such as at least one exterior surface comprising a plurality of hydroxyl polymer filaments, such that the layered fibrous structures exhibit novel surface and/or softness properties.

In yet another example, the layered fibrous structures of the present invention may comprise a plurality of filaments, for example hydroxyl polymer filaments, such as at least one exterior surface comprising a plurality of hydroxyl polymer filaments, such that the layered fibrous structures exhibit novel surface and/or softness properties.

In yet another example, the layered fibrous structures of the present invention may be uncreped layered fibrous structures comprising a plurality of fibrous elements, for example hydroxyl polymer filaments, such as at least one exterior surface comprising a plurality of hydroxyl polymer filaments, such that the uncreped layered fibrous structures exhibit novel surface and/or softness properties. In another example of the present invention, a layered fibrous structure comprising a plurality of hydroxyl polymer filaments that are present in an exterior layer and/or on an exterior surface of the layered fibrous structure at a level of greater than 10 and/or greater than 12 and/or greater than 14 and/or greater than 16 and/or at least 18 and/or less than 40 and/or less than 35 and/or less than 30 and/or less than 25 gsm, such that the layered fibrous structure exhibits novel surface and/or softness properties.

In yet another example, the layered fibrous structures of the present invention may comprise a plurality of filaments, for example hydroxyl polymer filaments, such as at least one exterior surface comprising a plurality of hydroxyl polymer filaments, such that the layered fibrous structures exhibit novel surface and/or softness properties.

In addition, the layered fibrous structures of the present invention may exhibit any features of the above described layered fibrous structures of the present invention.

In addition, the layered fibrous structures of the present invention may be non-lotioned and/or may not contain a post-applied surface chemistry. In another example, the layered fibrous structures of the present invention may be creped or uncreped. In one example, the layered fibrous structures of the present invention are uncreped layered fibrous structures.

In addition to the layered fibrous structures of the present invention exhibiting improved surface and/or softness properties as described herein, such layered fibrous structures also may exhibit improved cleaning properties, for example bowel movement cleaning properties, compared to known layered fibrous structures, for example known layered fibrous structures comprising hydroxyl polymer filaments and known layered fibrous structures, such as wet-laid and/or air-laid, comprising cellulose fibers, for example pulp fibers. Without wishing to be bound by theory, it is believed that the layered fibrous structures of the present invention exhibit improved cleaning properties due to the hydroxyl polymer fibrous elements of the present invention exhibiting greater absorbency, without a gooey feel, than pulp fibers, and therefore facilitates better, in reality and/or perception, absorption of bowel movement and/or urine more completely and/or faster than known layered fibrous structures. In addition, it is believed that the layered fibrous structures of the present invention that comprise a plurality of hydroxyl polymer fibrous elements, for example hydroxyl polymer filaments in an exterior layer, such as a scrim, provides an improved adsorbency, without a gooey feel, than known layered fibrous structures, such that the hydroxyl polymer fibrous elements during use contact the user's skin surface and trap and/or lock in the bowel movement or portions thereof. Further, it is believed that the layered fibrous structures of the present invention that comprise a plurality of hydroxyl polymer fibrous elements, for example hydroxyl polymer filaments in an exterior layer, such as a scrim, that provide improved surface and/or softness properties permits a user to apply more force to the layered fibrous structure during use because the hydroxyl polymer fibrous elements provide a cushion and/or buffer compared to known layered fibrous structures, especially known wet-laid and/or air-laid layered fibrous structures that consist or consist essentially of pulp fibers.

In addition to the improved surface and/or softness properties, the layered fibrous structures of the present invention may also exhibit a novel glide value, that permits a more comfortable wiping experience when a user uses the layered fibrous structure to remove bowel movement and/or urine from the user's body, for example skin.

The layered fibrous structures of the present invention may be embossed and/or tufted that creates a three-dimensional surface pattern that provides aesthetics and/or improved cleaning properties. The level of improved cleaning properties relates to the % contact area under a load, such as a user's force applied to the layered fibrous structure during wiping, and/or % volume/area under a load, such as a user's force applied to the layered fibrous structure during wiping, created by the three-dimensional surface pattern on the surface of the layered fibrous structure. In one example, the emboss area may be greater than 10% and/or greater than 12% and/or greater than 15% and/or greater than 20% of the surface area of at least one surface of the layered fibrous structure. Even with such a high emboss area, the layered fibrous structures of the present invention still exhibit the improved surface and/or softness properties.

### Fibrous Elements

The fibrous elements of the present invention may be produced from a polymer melt composition, for example a hydroxyl polymer melt composition such as an aqueous hydroxyl polymer melt composition, comprising a hydroxyl polymer, such as an uncrosslinked starch for example a dent corn starch, an acid-thinned starch, and/or a starch derivative such as an ethoxylated starch, a crosslinking system comprising a crosslinking agent, such as an imidazolidinone, and water. In one example, the crosslinking agent comprises less than 2% and/or less than 1.8% and/or less than 1.5% and/or less than 1.25% and/or 0% and/or about 0.25% and/or about 0.50% by weight of a base, for example triethanolamine. It has unexpectedly been found that the reducing the level of base in the crosslinking agent used in the polymer melt composition results in more effective crosslinking. In one example, the fibrous elements of the present invention comprise greater than 25% and/or greater than 40% and/or greater than 50% and/or greater than 60% and/or greater than 70% to about 95% and/or to about 90% and/or to about 80% by weight of the fibrous element of a hydroxyl polymer, such as starch, which may be in a crosslinked state. In one example, the fibrous element comprises an ethoxylated starch and an acid thinned starch, which may be in their crosslinked states.

The fibrous elements may also comprise a crosslinking agent, such as an imidazolidinone, which may be in its crosslinked state (crosslinking the hydroxyl polymers present in the fibrous elements) at a level of from about 0.25% and/or from about 0.5% and/or from about 1% and/or from about 2% and/or from about 3% and/or to about 10% and/or to about 7% and/or to about 5.5% and/or to about 4.5% by weight of the fibrous element. In addition to the crosslinking agent, the fibrous element may comprise a crosslinking facilitator that aids the crosslinking agent at a level of from 0% and/or from about 0.3% and/or from about 0.5% and/or to about 2% and/or to about 1.7% and/or to about 1.5% by weight of the fibrous element.

In one example, the hydroxyl polymer fibrous element, for example hydroxyl polymer filament, comprises a crosslinked hydroxyl polymer, such as a crosslinked starch and/or starch derivative.

The polymer melt composition may also comprise a surfactant, such as a sulfosuccinate surfactant. A non-limiting example of a suitable sulfosuccinate surfactant comprises Aerosol® AOT (a sodium dioctyl sulfosuccinate) and/or Aerosol® MA-80 (a sodium dihexyl sulfosuccinate), which are commercially available from Cytec. The surfactant, such as a sulfosuccinate surfactant, may be present at a level of from 0% and/or from about 0.1% and/or from about 0.3% to about 2% and/or to about 1.5% and/or to about 1.1% and/or to about 0.7% by weight of the fibrous element.

In addition to the crosslinking agent, the polymer melt composition may comprise a crosslinking facilitator such as ammonium salts of methanesulfonic acid, ethanesulfonic acid, propanesulfonic acid, isopropylsulfonic acid, butanesulfonic acid, isobutylsulfonic acid, sec-butylsulfonic acids, benzenesulfonic acid, toluenesulfonic acid, xylenesulfonic acid, cumenesulfonic acid, alkylbenzenesulfonic, alkylnaphthalenedisulfonic acids.

The fibrous elements may also comprise a polymer selected from the group consisting of: polyacrylamide and its derivatives; acrylamide-based copolymers, polyacrylic acid, polymethacrylic acid, and their esters; polyethyleneimine; copolymers made from mixtures of monomers of the aforementioned polymers; and mixtures thereof at a level of from 0% and/or from about 0.01% and/or from about 0.05% and/or to about 0.5% and/or to about 0.3% and/or to about 0.2% by weight of the fibrous element. Such polymers may exhibits a weight average molecular weight of greater than 500,000 g/mol. In one example, the fibrous element comprises polyacrylamide.

The fibrous elements may also comprise various other ingredients such as propylene glycol, sorbitol, glycerin, and mixtures thereof.

One or more hueing agents, such as Violet CT may also be present in the polymer melt composition and/or fibrous elements formed therefrom.

In one example, the fibrous elements, of the present invention comprise a fibrous element-forming polymer, such as a hydroxyl polymer, for example a crosslinked hydroxyl polymer. In one example, the fibrous elements may comprise two or more fibrous element-forming polymers, such as two or more hydroxyl polymers. In another example, the fibrous element may comprise two or more fibrous element-forming polymers, such as two or more hydroxyl polymers, at least one of which is starch and/or a starch derivative. In still another example, the fibrous elements of the present invention may comprise two or more fibrous element-forming polymers at least one of which is a hydroxyl polymer and at least one of which is a non-hydroxyl polymer.

In yet another example, the fibrous elements of the present invention may comprise two or more non-hydroxyl polymers. In one example, at least one of the non-hydroxyl polymers exhibits a weight average molecular weight of greater than 1,400,000 g/mol and/or is present in the fibrous elements at a concentration greater than its entanglement concentration (Cₑ) and/or exhibits a polydispersity of greater than 1.32. In still another example, at least one of the non-hydroxyl polymers comprises an acrylamide-based copolymer.

In one example, the fibrous element comprises a filament. In another example, the fibrous element comprises a fiber, such as a filament that has been cut into fibers.

### Fibrous Element-Forming Polymers

The polymer melt compositions of the present invention, for example hydroxyl polymer melt compositions such as aqueous hydroxyl polymer melt compositions, and/or fibrous elements, such as filaments and/or fibers, of the present invention that associate to form layered fibrous structures of the present invention contain at least one fibrous element-forming polymer, such as a hydroxyl polymer, and may contain other types of polymers such as non-hydroxyl polymers that exhibit weight average molecular weights of greater than 500,000 g/mol and mixtures thereof.

Non-limiting examples of hydroxyl polymers in accordance with the present invention include polyols, such as polyvinyl alcohol, polyvinyl alcohol derivatives, polyvinyl alcohol copolymers, starch, starch derivatives, starch copolymers, chitosan, chitosan derivatives, chitosan copolymers, cellulose, cellulose derivatives such as cellulose ether and ester derivatives, cellulose copolymers, hemicellulose, hemicellulose derivatives, hemicellulose copolymers, gums, arabinans, galactans, proteins and various other polysaccharides and mixtures thereof.

In one example, a hydroxyl polymer of the present invention comprises a polysaccharide.

In another example, a hydroxyl polymer of the present invention comprises a non-thermoplastic polymer.

The hydroxyl polymer may have a weight average molecular weight of from about 10,000 g/mol to about 40,000,000 g/mol and/or greater than 100,000 g/mol and/or greater than 1,000,000 g/mol and/or greater than 3,000,000 g/mol and/or greater than 3,000,000 g/mol to about 40,000,000 g/mol. Higher and lower molecular weight hydroxyl polymers may be used in combination with hydroxyl polymers having a certain desired weight average molecular weight.

Polyvinyl alcohols herein can be grafted with other monomers to modify its properties. A wide range of monomers has been successfully grafted to polyvinyl alcohol. Non-limiting examples of such monomers include vinyl acetate, styrene, acrylamide, acrylic acid, 2-hydroxyethyl methacrylate, acrylonitrile, 1,3-butadiene, methyl methacrylate, methacrylic acid, vinylidene chloride, vinyl chloride, vinyl amine and a variety of acrylate esters. Polyvinyl alcohols comprise the various hydrolysis products formed from polyvinyl acetate. In one example the level of hydrolysis of the polyvinyl alcohols is greater than 70% and/or greater than 88% and/or greater than 95% and/or about 99%.

“Polysaccharides” as used herein means natural polysaccharides and polysaccharide derivatives and/or modified polysaccharides. Suitable polysaccharides include, but are not limited to, starches, starch derivatives, starch copolymers, chitosan, chitosan derivatives, chitosan copolymers, cellulose, cellulose derivatives, cellulose copolymers, hemicellulose, hemicellulose derivatives, hemicelluloses copolymers, gums, arabinans, galactans, and mixtures thereof. The polysaccharide may exhibit a weight average molecular weight of from about 10,000 to about 40,000,000 g/mol and/or greater than about 100,000 and/or greater than about 1,000,000 and/or greater than about 3,000,000 and/or greater than about 3,000,000 to about 40,000,000.

The polysaccharides of the present invention may comprise non-cellulose and/or non-cellulose derivative and/or non-cellulose copolymer hydroxyl polymers. Non-limiting example of such non-cellulose polysaccharides may be selected from the group consisting of: starches, starch derivatives, starch copolymers, chitosan, chitosan derivatives, chitosan copolymers, hemicellulose, hemicellulose derivatives, hemicelluloses copolymers, and mixtures thereof.

In one example, the hydroxyl polymer comprises starch, a starch derivative and/or a starch copolymer. In another example, the hydroxyl polymer comprises starch and/or a starch derivative. In yet another example, the hydroxyl polymer comprises starch. In one example, the hydroxyl polymer comprises ethoxylated starch. In another example, the hydroxyl polymer comprises acid-thinned starch. In still another example, the hydroxyl polymer comprises Dent corn starch.

As is known, a natural starch can be modified chemically or enzymatically, as well known in the art. For example, the natural starch can be acid-thinned, hydroxy-ethylated, hydroxy-propylated, ethersuccinylated or oxidized. In one example, the starch comprises a high amylopectin natural starch (a starch that contains greater than 75% and/or greater than 90% and/or greater than 98% and/or about 99% amylopectin). Such high amylopectin natural starches may be derived from agricultural sources, which offer the advantages of being abundant in supply, easily replenishable and relatively inexpensive. Chemical modifications of starch typically include acid or alkaline-catalyzed hydrolysis and chain scission (oxidative and/or enzymatic) to reduce molecular weight and molecular weight distribution. Suitable compounds for chemical modification of starch include organic acids such as citric acid, acetic acid, glycolic acid, and adipic acid; inorganic acids such as hydrochloric acid, sulfuric acid, nitric acid, phosphoric acid, boric acid, and partial salts of polybasic acids, e.g., KH₂PO₄, NaHSO₄; group Ia or IIa metal hydroxides such as sodium hydroxide, and potassium hydroxide; ammonia; oxidizing agents such as hydrogen peroxide, benzoyl peroxide, ammonium persulfate, potassium permanganate, hypochloric salts, and the like; and mixtures thereof.

“Modified starch” is a starch that has been modified chemically or enzymatically. The modified starch is contrasted with a native starch, which is a starch that has not been modified, chemically or otherwise, in any way.

Chemical modifications may also include derivatization of starch by reaction of its hydroxyl groups with alkylene oxides, and other ether-, ester-, urethane-, carbamate-, or isocyanate-forming substances. Hydroxyalkyl, ethersuccinylated, acetyl, or carbamate starches or mixtures thereof can be used as chemically modified starches. The degree of substitution of the chemically modified starch is from 0.001 to 3.0, and more specifically from 0.003 to 0.2. Biological modifications of starch may include bacterial digestion of the carbohydrate bonds, or enzymatic hydrolysis using enzymes such as amylase, amylopectase, and the like.

Generally, all kinds of natural starches can be used in the present invention. Suitable naturally occurring starches can include, but are not limited to: corn starch, potato starch, sweet potato starch, wheat starch, sago palm starch, tapioca starch, rice starch, soybean starch, arrow root starch, amioca starch, bracken starch, lotus starch, waxy maize starch, and high amylose corn starch. Naturally occurring starches, particularly corn starch and wheat starch, can be particularly beneficial due to their low cost and availability.

In one example, to generate rheological properties suitable for high-speed fibrous element spinning processes, the molecular weight of the natural, unmodified starch may be reduced. The optimum molecular weight is dependent on the type of starch used. For example, a starch with a low level of amylose component, such as a waxy maize starch, disperses rather easily in an aqueous solution with the application of heat and does not retrograde or recrystallize significantly. With these properties, a waxy maize starch can be used at a weight average molecular weight, for example in the range of 500,000 g/mol to 40,000,000 g/mol. Modified starches such as hydroxy-ethylated Dent corn starch, which contains about 25% amylose, or oxidized Dent corn starch tend to retrograde more than waxy maize starch but less than acid thinned starch. This retrogradation, or recrystallization, acts as a physical cross-linking to effectively raise the weight average molecular weight of the starch in aqueous solution. Therefore, an appropriate weight average molecular weight for a typical commercially available hydroxyethylated Dent corn starch with 2 wt. % hydroxyethylation or oxidized Dent corn starch is from about 200,000 g/mol to about 10,000,000 g/mol. For ethoxylated starches with higher degrees of ethoxylation, for example a hydroxyethylated Dent corn starch with 5 wt % hydroxyethylation, weight average molecular weights of up to 40,000,000 g/mol may be suitable for the present invention. For acid thinned Dent corn starch, which tends to retrograde more than oxidized Dent corn starch, the appropriate weight average molecular weight is from about 100,000 g/mol to about 15,000,000 g/mol.

The weight average molecular weight of starch may also be reduced to a desirable range for the present invention by physical/mechanical degradation (e.g., via the thermomechanical energy input of the processing equipment).

The natural starch can be hydrolyzed in the presence of an acid catalyst to reduce the molecular weight and molecular weight distribution of the composition. The acid catalyst can be selected from the group consisting of hydrochloric acid, sulfuric acid, phosphoric acid, citric acid, ammonium chloride and any combination thereof. Also, a chain scission agent may be incorporated into a spinnable starch composition such that the chain scission reaction takes place substantially concurrently with the blending of the starch with other components. Non-limiting examples of oxidative chain scission agents suitable for use herein include ammonium persulfate, hydrogen peroxide, hypochlorite salts, potassium permanganate, and mixtures thereof. Typically, the chain scission agent is added in an amount effective to reduce the weight average molecular weight of the starch to the desirable range. It is found that compositions having modified starches in the suitable weight average molecular weight ranges have suitable shear viscosities, and thus improve processability of the composition. The improved processability is evident in less interruptions of the process (e.g., reduced breakage, shots, defects, hang-ups) and better surface appearance and strength properties of the final product, such as fibers of the present invention.

In one example, the fibrous element of the present invention is void of thermoplastic, water-insoluble polymers.

In one example, the fibrous element-forming polymers may be present in the aqueous hydroxyl polymer melt composition at an amount of from about 20% to about 50% and/or from about 30% to about 50% and/or from about 35% to about 48% by weight of the aqueous hydroxyl polymer melt composition and present in a polymeric structure, for example fibrous element and/or layered fibrous structure, at a level of from about 50% to about 100% and/or from about 60% to about 98% and/or from about 75% to about 95% by weight of the polymeric structure, for example fibrous element and/or layered fibrous structure.

### Other Polymers

The polymer melt compositions of the present invention and/or fibrous elements, such as filaments of the present invention may comprise, in addition to the fibrous element-forming polymer, other polymers, such as non-hydroxyl polymers.

Non-limiting examples of suitable non-hydroxyl polymers that may be included in the fibrous elements of the present invention include non-hydroxyl polymers that exhibit a weight average molecular weight of greater than 500,000 g/mol and/or greater than 750,000 g/mol and/or greater than 1,000,000 g/mol and/or greater than 1,250,000 g/mol and/or at greater than 1,400,000 g/mol and/or at least 1,450,000 g/mol and/or at least 1,500,000 g/mol and/or less than 10,000,000 g/mol and/or less than 5,000,000 g/mol and/or less than 2,500.00 g/mol and/or less than 2,000,000 g/mol and/or less than 1,750,000 g/mol.

In one example, the non-hydroxyl polymer exhibits a polydispersity of greater than 1.10 and/or at least 1.20 and/or at least 1.30 and/or at least 1.32 and/or at least 1.40 and/or at least 1.45.

Non-limiting examples of suitable non-hydroxyl polymers include polyacrylamide and derivatives such as carboxyl modified polyacrylamide polymers and copolymers including polyacrylic, poly(hydroxyethyl acrylic), polymethacrylic acid and their partial esters; vinyl polymers including polyvinylalcohol, polyvinylpyrrolidone, and the like; polyamides; polyalkylene oxides such as polyethylene oxide and mixtures thereof. Copolymers or graft copolymers made from mixtures of monomers selected from the aforementioned polymers are also suitable herein. Non-limiting examples of commercially available polyacrylamides include nonionic polyacrylamides such as N300 from Kemira or Hyperfloc® NF221, NF301, and NF241 from Hychem, Inc.

In one example, the non-hydroxyl polymers may be present in an amount of from about 0.01% to about 10% and/or from about 0.05% to about 5% and/or from about 0.075% to about 2.5% and/or from about 0.1% to about 1%, by weight of the aqueous hydroxyl polymer melt composition, filament and/or layered fibrous structure.

In yet another example, the non-hydroxyl polymer comprises a linear polymer. In another example, the non-hydroxyl polymer comprises a long chain branched polymer. In still another example, the non-hydroxyl polymer is compatible with the hydroxyl polymer at a concentration greater than the non-hydroxyl polymer's entanglement concentration Cₑ.

Non-limiting examples of suitable non-hydroxyl polymers are selected from the group consisting of: polyacrylamide and its derivatives; polyacrylic acid, polymethacrylic acid and their esters; polyethyleneimine; copolymers made from mixtures of the aforementioned polymers; and mixtures thereof. In one example, the non-hydroxyl polymer comprises polyacrylamide. In one example, the fibrous elements comprises two or more non-hydroxyl polymers, such as two or more polyacrylamides, such at two or more different weight average molecular weight polyacrylamides.

In one example, the non-hydroxyl polymer comprises an acrylamide-based copolymer. In another example, the non-hydroxyl polymer comprises a polyacrylamide and an acrylamide-based copolymer. In one example, the acrylamide-based copolymer is derived from an acrylamide monomer and at least one monomer selected from the group consisting of: pendant hydroxyl-containing monomers, pendant hydroxyl alkylether-containing monomers, pendant hydroxyl alkylester-containing monomers, pendant hydroxyl alkylamide-containing monomers, and mixtures thereof. In one example, the acrylamide-based copolymer comprises an acrylamide monomeric unit and at least one monomeric unit selected from the group consisting of: pendant hydroxyl-containing monomeric units, pendant hydroxyl alkylether-containing monomeric units, pendant hydroxyl alkylester-containing monomeric units, pendant hydroxyl alkylamide-containing monomeric units, and mixtures thereof.

### Crosslinking System

A crosslinking system comprising a crosslinking agent, such as an imidazolidinone, and optionally, a crosslinking facilitator, such as an ammonium salt, may be present in the polymer melt composition and/or may be added to the polymer melt composition before polymer processing of the polymer melt composition.

“Crosslinking agent” as used herein means any material that is capable of crosslinking a hydroxyl polymer within a polymer melt composition according to the present. Non-limiting examples of suitable crosslinking agents include polycarboxylic acids and/or imidazolidinones, such as dihydroxyethyleneurea (DHEU). In one example, the crosslinking agent is in the form of a solution rather than a recrystallized form. In another example, the crosslinking agent comprises less than 2% and/or less than 1.8% and/or less than 1.5% and/or less than 1.25% and/or 0% and/or to about 0.25% and/or to about 0.5% by weight of a base, such as triethanolamine.

“Crosslinking facilitator” as used herein means any material that is capable of activating a crosslinking agent thereby transforming the crosslinking agent from its unactivated state to its activated state.

Upon crosslinking the hydroxyl polymer during the curing step, the crosslinking agent becomes an integral part of the polymeric structure as a result of crosslinking the hydroxyl polymer as shown in the following schematic representation:
Hydroxyl polymer-Crosslinking agent-Hydroxyl polymer

The crosslinking facilitator may include derivatives of the material that may exist after the transformation/activation of the crosslinking agent. For example, a crosslinking facilitator salt being chemically changed to its acid form and vice versa.

Non-limiting examples of suitable crosslinking facilitators include acids having a pKa of less than 6 or salts thereof. The crosslinking facilitators may be Bronsted Acids and/or salts thereof, such as ammonium salts thereof.

In addition, metal salts, such as magnesium and zinc salts, can be used alone or in combination with Bronsted Acids and/or salts thereof, as crosslinking facilitators.

Non-limiting examples of suitable crosslinking facilitators include benzoic acid, citric acid, formic acid, glycolic acid, lactic acid, maleic acid, phthalic acid, phosphoric acid, hypophosphoric acid, succinic acid, and mixtures thereof and/or their salts, such as their ammonium salts, such as ammonium glycolate, ammonium citrate, ammonium chloride, ammonium sulfate.

Additional non-limiting examples of suitable crosslinking facilitators include glyoxal bisulfate salts, primary amine salts, such as hydroxyethyl ammonium salts, hydroxypropyl ammonium salt, secondary amine salts, ammonium toluene sulfonate, ammonium benzene sulfonate, ammonium xylene sulfonate, magnesium chloride, and zinc chloride.

### Surfactants

The polymer melt compositions of the present invention and/or fibrous elements of the present invention and layered fibrous structures formed thereform may comprise one or more surfactants. In one example, the surfactant is a fast wetting surfactant. In another example, the surfactant comprises a non-fast wetting surfactant, such as Aerosol® OT from Cytec.

Non-limiting examples of suitable fast wetting surfactants include surfactants that exhibit a twin-tailed general structure, for example a surfactant that exhibits a structure VIIA or VIIB as follows.

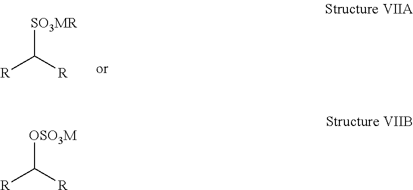

wherein R is independently selected from substituted or unsubstituted, linear or branched aliphatic groups and mixtures thereof. In one example, R is independently selected from substituted or unsubstituted, linear or branched C₄-C₇ aliphatic chains and mixtures thereof. In another example, R is independently selected from substituted or unsubstituted, linear or branched C₄-C₇ alkyls and mixtures thereof and M is a suitable cation, such as an alkali metal cation and/or an ammonium cation. In another example, R is independently selected from substituted or unsubstituted, linear or branched C₅-C₆ alkyls and mixtures thereof. In still another example, R is independently selected from substituted or unsubstituted, linear or branched C₆ alkyls and mixtures thereof. In even another example, R is an unsubstituted, branched C₆ alkyl having the following structure VIII.

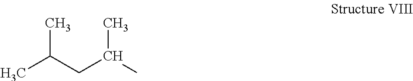

In another example, R is independently selected from substituted or unsubstituted, linear or branched C₅ alkyls and mixtures thereof. In yet another example, R is independently selected from unsubstituted, linear C₅ alkyls and mixtures thereof. The C₅ alkyl may comprise a mixture of unsubstituted linear C₅ alkyls, for example C₅ n-pentyl, and/or 1-methyl branched C₅ alkyls as shown in the following structure IX.

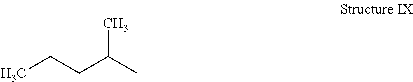

In even another example, R comprises a mixture of C₄-C₇ alkyls and/or a mixture of C₅-C₆ alkyls.

The fast wetting surfactants may be present in the polymer melt compositions, fibrous elements, and/or layered fibrous structures of the present invention, alone or in combination with other non-fast wetting surfactants.

In one example, the fast wetting surfactants of the present invention may be used individually or in mixtures with each other or in a mixture with one or more non-fast wetting surfactants, for example a C₈ sulfosuccinate surfactant where R is the following structure X

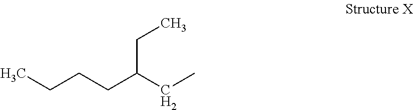

In one example a fast wetting surfactant comprises a sulfosuccinate surfactant having the following structure XI.

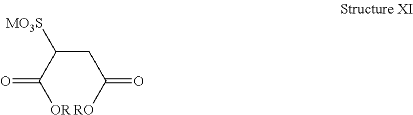

wherein R is independently selected from substituted or unsubstituted, linear or branched aliphatic groups and mixtures thereof and M is a suitable cation, such as an alkali metal cation and/or an ammonium cation. In one example, R is independently selected from substituted or unsubstituted, linear or branched C₄-C₇ aliphatic chains and mixtures thereof. In another example, R is independently selected from substituted or unsubstituted, linear or branched C₄-C₇ alkyls and mixtures thereof. In another example, R is independently selected from substituted or unsubstituted, linear or branched C₅-C₆ alkyls and mixtures thereof. In still another example, R is independently selected from substituted or unsubstituted, linear or branched C₆ alkyls and mixtures thereof. In even another example, R is an unsubstituted, branched C₆ alkyl having the following structure XII.

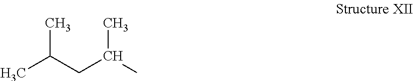

Non-limiting examples of fast wetting surfactants according to the present invention include sulfosuccinate surfactants, for example a sulfosuccinate surfactant that has structure VIII as its R groups (Aerosol® MA-80), a sulfosuccinate surfactant that has C₄ isobutyl as its R groups (Aerosol® IB), and a sulfosuccinate surfactant that has a mixture of C₅ n-pentyl and structure IX as its R groups (Aerosol® AY), all commercially available from Cytec.

Additional non-limiting examples of fast wetting surfactants according to the present invention include alcohol sulfates derived from branched alcohols such as Isalchem and Lial alcohols (from Sasol) ie. Dacpon 27 23 AS and Guerbet alcohols from Lucky Chemical. Still another example of a fast wetting surfactant includes paraffin sulfonates such as Hostapur SAS30 from Clariant.

Typically, the fast wetting surfactants are present in an amount of from about 0.01% to about 5% and/or from about 0.5% to about 2.5% and/or from about 1% to about 2% and/or from about 1% to about 1.5%, by weight of the polymer melt composition, fibrous element and/or layered fibrous structure.

A fast wetting surfactant may be present both in the interior and exterior of the fibrous elements produced from the polymer melt composition, which is distinguished from a surface only treatment of the formed fibrous elements. Any fast wetting surfactant that is present on the exterior of a fibrous element may be determined by extracting the fibrous element with a solvent that dissolves the surfactant, but does not swell the fibrous element and then analyzing for the surfactant by LC-mass spec. The surfactant that is present in the interior of the fibrous element may be determined by extracting the fibrous element with a solvent that dissolves the surfactant and also swells the fibrous elements, such as water/alcohol or water/acetone mixtures followed by analysis for surfactant by a technique such as LC mass spec. Alternatively, the fibrous element may be treated with an enzyme such as amylase that degrades the fibrous element-forming polymer, for example polysaccharide, but not the fast wetting surfactant and the resulting solution may be analyzed for the surfactant by LC-mass spec.

### Hueing Agents

The polymer melt compositions and/or fibrous elements of the present invention may comprise one or more hueing agents. In one example, the total level of one or more hueing agents present within one or more, for example a plurality, of the fibrous elements of a layered fibrous structure of the present invention is less than 1% and/or less than 0.5% and/or less than 0.05% and/or less than 0.005% and/or greater than 0.00001% and/or greater than 0.0001% and/or greater than 0.001% by weight of the dry fibrous element and/or dry layered fibrous structure formed by fibrous elements containing the hueing agents. In one example, the total level of one or more hueing agents present within one or more, for example a plurality, of the fibrous elements of a layered fibrous structure of the present invention is from about 0.0001% to about 0.5% and/or from about 0.0005% to about 0.05% and/or from about 0.001% to about 0.05% and/or from about 0.001% to about 0.005% by weight of the dry fibrous element and/or dry layered fibrous structure formed by fibrous elements containing the hueing agents. Hueing agents can be used either alone or in combination. Hueing agents may be selected from any known chemical class of dye, including but not limited to acridine, anthraquinone (including polycyclic quinones), azine, azo (e.g., monoazo, disazo, trisazo, tetrakisazo, polyazo), including premetallized azo, benzodifurane and benzodifuranone, carotenoid, coumarin, cyanine, diazahemicyanine, diphenylmethane, formazan, hemicyanine, indigoids, methane, naphthalimides, naphthoquinone, nitro and nitroso, oxazine, phthalocyanine, pyrazoles, stilbene, styryl, triarylmethane, triphenylmethane, xanthenes and mixtures thereof.

Non-limiting examples of hueing agents include dyes, dye-clay conjugates, and organic and inorganic pigments and mixtures thereof. Suitable dyes include small molecule dyes and polymeric dyes. Suitable small molecule dyes include small molecule dyes selected from the group consisting of dyes falling into the Colour Index (C.I.) classifications of Direct, Basic, Reactive or hydrolysed Reactive, Solvent or Disperse dyes for example that are classified as Blue, Violet, Red, Green or Black, and mixtures thereof. In another aspect, suitable small molecule dyes include small molecule dyes selected from the group consisting of Colour Index (Society of Dyers and Colourists, Bradford, UK) numbers Direct Violet dyes such as 9, 35, 48, 51, 66, and 99, Direct Blue dyes such as 1, 71, 80 and 279, Acid Red dyes such as 17, 73, 52, 88 and 150, Acid Violet dyes such as 15, 17, 24, 43, 49 and 50, Acid Blue dyes such as 15, 17, 25, 29, 40, 45, 75, 80, 83, 90 and 113, Acid Black dyes such as 1, Basic Violet dyes such as 1, 3, 4, 10 and 35, Basic Blue dyes such as 3, 16, 22, 47, 66, 75 and 159, Disperse or Solvent dyes such as those described in US 2008/034511 A1 or U.S. Pat. No. 8,268,016 B2, or dyes as disclosed in U.S. Pat. No. 7,208,459 B2, and mixtures thereof. In another aspect, suitable small molecule dyes include small molecule dyes selected from the group consisting of C.I. Acid Violet 17, Direct Blue 71, Direct Violet 51, Direct Blue 1, Acid Red 88, Acid Red 150, Acid Blue 29, Acid Blue 113 or mixtures thereof.

Suitable polymeric dyes include polymeric dyes selected from the group consisting of polymers containing covalently bound (sometimes referred to as conjugated) chromogens, (dye-polymer conjugates), for example polymers with chromogens co-polymerized into the backbone of the polymer and mixtures thereof. Polymeric dyes include those described in WO2011/98355, US 2012/225803 A1, US 2012/090102 A1, U.S. Pat. No. 7,686,892 B2, and WO2010/142503.

In another aspect, suitable polymeric dyes include polymeric dyes selected from the group consisting of hueing agents commercially available under the trade name of Liquitint® (Milliken, Spartanburg, S.C., USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® Violet CT, carboxymethyl cellulose (CMC) covalently bound to a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colourants, alkoxylated thiophene polymeric colourants, and mixtures thereof.

### Solid Additives

The layered fibrous structures of the present invention may further comprise one or more solid additives. “Solid additive” as used herein means an additive that is capable of being applied to a surface of a layered fibrous structure and/or nonwoven substrate of the layered fibrous structure in a solid form. In other words, the solid additive of the present invention can be delivered directly to a surface of the layered fibrous structure and/or nonwoven substrate of the layered fibrous structure without a liquid phase being present, i.e. without melting the solid additive and without suspending the solid additive in a liquid vehicle or carrier. As such, the solid additive of the present invention does not require a liquid state or a liquid vehicle or carrier in order to be delivered to a surface of a nonwoven substrate. The solid additive of the present invention may be delivered via a gas or combinations of gases. In one example, in simplistic terms, a solid additive is an additive that when placed within a container, does not take the shape of the container. In one example, a solid additive comprises a naturally occurring fiber, such as a pulp fiber.

The solid additives of the present invention may have different geometries and/or cross-sectional areas that include round, elliptical, star-shaped, rectangular, trilobal and other various eccentricities.

In one example, the solid additive may exhibit a particle size of less than 6 mm and/or less than 5.5 mm and/or less than 5 mm and/or less than 4.5 mm and/or less than 4 mm and/or less than 2 mm in its maximum dimension.

“Particle” as used herein means an object having an aspect ratio of less than about 25/1 and/or less than about 15/1 and/or less than about 10/1 and/or less than 5/1 to about 1/1. A particle is not a fiber as defined herein.

The solid additives may be present in the layered fibrous structures of the present invention at a level of greater than about 1 and/or greater than about 2 and/or greater than about 4 and/or to about 20 and/or to about 15 and/or to about 10 g/m². In one example, a layered fibrous structure of the present invention comprises from about 2 to about 10 and/or from about 5 to about 10 g/m² of solid additive.

In one example, the solid additives are present in the layered fibrous structures of the present invention at a level of greater than 5% and/or greater than 10% and/or greater than 20% to about 50% and/or to about 40% and/or to about 30%.

### Scrim Material

The layered fibrous structures of the present invention may further comprise a scrim material. The scrim material may comprise any suitable material capable of bonding to a nonwoven substrate of the layered fibrous structure of the present invention. In one example, the scrim material comprises a material that can be thermally bonded to the nonwoven substrate of the layered fibrous structure of the present invention. Non-limiting examples of suitable scrim materials include filaments of the present invention. In one example, the scrim material comprises filaments that comprise hydroxyl polymers. In another example, the scrim material comprises starch filaments. In yet another example, the scrim material comprises filaments comprising a thermoplastic polymer. In still another example, the scrim material comprises a layered fibrous structure according to the present invention wherein the layered fibrous structure comprises filaments comprising hydroxyl polymers, such as starch filaments, and/or thermoplastic polymers. In another example, the scrim material may comprise a film. In another example, the scrim material may comprise a nonwoven substrate according to the present invention. In even another example, the scrim material may comprise a latex.

In one example, the scrim material may be the same composition as the nonwoven substrate of the layered fibrous structure.

The scrim material and/or an exterior layer comprising a plurality of hydroxyl polymer fibrous elements of the layered fibrous structure may be present in the layered fibrous structures of the present invention at a basis weight of greater than 6 and/or greater than 8 and/or greater than 10 and/or greater than 12 and/or greater than 14 and/or greater than 16 and/or greater than 18 and/or less than 40 and/or less than 35 and/or less than 30 and/or less than 25 g/m² (“gsm”) and/or from about 12 to about 40 g/m² and/or from about 12 to about 35 g/m² and/or from about 12 to about 30 g/m² and/or from about 16 to about 25 g/m². The basis weight of the scrim material and/or exterior layer or other layers of the layered fibrous structure are known by the manufacturer when making the layered fibrous structure and may be determined by other means such as tape stripping or other suitable means known to those in the art.

### Methods for Making Layered Fibrous Structure

The methods of the present invention relate to producing layered fibrous structures comprising fibrous elements produced from polymer melt compositions comprising a fibrous element-forming polymer, such as a hydroxyl polymer, a crosslinking agent, an acrylamide-based copolymer, and other materials as described herein.

FIGS. 2 and 3 illustrate one example of a method for making a layered fibrous structure of the present invention. As shown in FIGS. 2 and 3, the method 10 comprises the steps of:

a. providing first hydroxyl polymer fibrous elements, for example hydroxyl polymer filaments, 12 from a first source 14 of hydroxyl polymer fibrous elements, which form a first layer 16 of hydroxyl polymer fibrous elements;

b. providing second hydroxyl polymer fibrous elements, for example hydroxyl polymer filaments, 18 from a second source 20 of hydroxyl polymer fibrous elements, which form a second layer 22 of hydroxyl polymer fibrous elements;

c. providing third hydroxyl polymer fibrous elements, for example hydroxyl polymer filaments, 24 from a third source 26 of hydroxyl polymer fibrous elements, which form a third layer 28 of hydroxyl polymer fibrous elements;

d. providing solid additives 30 from a source 32 of solid additives;

e. providing fourth hydroxyl polymer fibrous elements, for example hydroxyl polymer filaments, 34 from a fourth source 36 of hydroxyl polymer fibrous elements, which form a fourth layer 38 of hydroxyl polymer fibrous elements; and

f. collecting the first, second, third, and fourth hydroxyl polymer fibrous elements 12, 18, 24, 34 and the solid additives 30 to form a layered fibrous structure 40, wherein the first source 14 of hydroxyl polymer fibrous elements is oriented at a first angle α to the machine direction of the layered fibrous structure 40, the second source 20 of hydroxyl polymer fibrous elements is oriented at a second angle β to the machine direction different from the first angle α, the third source 26 is oriented at a third angle δ to the machine direction different from the first angle α and the second angle β, and wherein the fourth source 36 is oriented at a fourth angle ε to the machine direction different from the second angle β and third angle δ.

The first, second, and third layers 16, 22, 28 of hydroxyl polymer fibrous elements are collected on a collection device 42, which may be a belt or fabric, with or without the aid of a vacuum box 47. The collection device 42 may be a patterned belt that imparts a pattern, such as a non-random, repeating pattern to the layered fibrous structure 40 during the layered fibrous structure making process. The first, second, and third layers 16, 22, 28 of hydroxyl polymer fibrous elements are collected (for example one on top of the other) on the collection device 42 to form a multi-layer nonwoven substrate 44 upon which the solid additives 30 are deposited. The fourth layer 38 of hydroxyl polymer fibrous elements may then be deposited onto the solid additives 30 to form a scrim 46.

The first angle α and the fourth angle ε may be the same angle, for example 90° to the machine direction.

The second angle β and the third angle δ may be the same angle, just positive and negative of one another. For example the second angle β may be −40° to the machine direction and the third angle δ may be +40° to the machine direction.

In one example, at least one of the first, second, and third angles α, β, δ is less than 90° to the machine direction. In another example, the first angle α and/or fourth angle ε is about 90° to the machine direction. In still another example, the second angle β and/or third angle δ is from about ±10° to about ±80° and/or from about ±30° to about ±60° to the machine direction and/or about ±40° to the machine direction.

In one example, the first, second, and third layers 16, 22, 28 of filaments may be formed into a nonwoven substrate 44 prior to being utilized in the process for making a layered fibrous structure described above. In this case, the nonwoven substrate 44 would likely be in a parent roll that could be unwound into the layered fibrous structure making process and the solid additives 30 could be deposited directly onto a surface of the nonwoven substrate 44.

In one example, the step of providing a plurality of solid additives 30 onto the nonwoven substrate 44 may comprise airlaying the solid additives 30 using an airlaying former. A non-limiting example of a suitable airlaying former is available from Dan-Web of Aarhus, Denmark.

In one example, the step of providing fourth filaments 34 such that the filaments contact the solid additives 30 comprises the step of depositing the fourth filaments 34 such that at least a portion (in one example all or substantially all) of the solid additives 30 are contacted by the fourth filaments 34 thus positioning the solid additives 30 between the fourth layer 38 of filaments and the nonwoven substrate 44. Once the fourth layer 38 of filaments is in place, the layered fibrous structure 40 may be subjected to a bonding step that bonds the fourth layer 38 of filaments (in this case, the scrim 46) to the nonwoven substrate 44. This step of bonding may comprise a thermal bonding operation. The thermal bonding operation may comprise passing the layered fibrous structure 40 through a nip formed by thermal bonding rolls 48, 50. At least one of the thermal bonding rolls 48, 50 may comprise a pattern that is translated into the bond sites 52 formed in the layered fibrous structure 40.

In addition to being subjected to a bonding operation, the layered fibrous structure may also be subjected to other post-processing operations such as embossing, tuft-generating, gear rolling, which includes passing the layered fibrous structure through a nip formed between two engaged gear rolls, moisture-imparting operations, free-fiber end generating, and surface treating to form a finished layered fibrous structure. In one example, the layered fibrous structure is subjected to gear rolling by passing the layered fibrous structure through a nip formed by at least a pair of gear rolls. In one example, the layered fibrous structure is subjected to gear rolling such that free-fiber ends are created in the layered fibrous structure. The gear rolling may occur before or after two or more layered fibrous structures are combined to form a multi-ply sanitary tissue product. If it occurs after, then the multi-ply sanitary tissue product is passed through the nip formed by at least a pair of gear rolls.

The method for making a layered fibrous structure of the present invention may be close coupled (where the layered fibrous structure is convolutedly wound into a roll prior to proceeding to a converting operation) or directly coupled (where the layered fibrous structure is not convolutedly wound into a roll prior to proceeding to a converting operation) with a converting operation to emboss, print, deform, surface treat, or other post-forming operation known to those in the art. For purposes of the present invention, direct coupling means that the layered fibrous structure can proceed directly into a converting operation rather than, for example, being convolutedly wound into a roll and then unwound to proceed through a converting operation.

In one example, one or more plies of the layered fibrous structure according to the present invention may be combined, for example with glue, with another ply of layered fibrous structure, which may also be a layered fibrous structure according to the present invention, to form a multi-ply sanitary tissue product. In one example, the multi-ply sanitary tissue product may be formed by combining two or more plies of layered fibrous structure according to the present invention. In another example, two or more plies of layered fibrous structure according to the present invention may be combined to form a multi-ply sanitary tissue product such that the solid additives present in the layered fibrous structure plies are adjacent to each of the outer surfaces of the multi-ply sanitary tissue product.

The process of the present invention may include preparing individual rolls of layered fibrous structure and/or sanitary tissue product comprising such layered fibrous structure(s) that are suitable for consumer use.

Even though the above non-limiting example of a process according to the present invention describes the use of four sources of filaments and four layers of filaments, the process may utilize a single source of fibrous elements and/or multiple (greater than 4) sources of fibrous elements and the layered fibrous structure may be a single layer or multiple layers depending on the number of sources of fibrous elements and/or sources of solid additives.

In one example, the sources of filaments comprise meltblow dies that produce filaments from a polymer melt composition according to the present invention. In one example, as shown in FIG. 4 the meltblow die 54 may comprise at least one fibrous element-forming hole 56, and/or 2 or more and/or 3 or more rows of fibrous element-forming holes 56 from which filaments are spun. At least one row of the fibrous element-forming holes 56 contains 2 or more and/or 3 or more and/or 10 or more fibrous element-forming holes 56. In addition to the fibrous element-forming holes 56, the meltblow die 54 comprises fluid-releasing holes 58, such as gas-releasing holes, in one example air-releasing holes, that provide attenuation to the filaments formed from the fibrous element-forming holes 56. One or more fluid-releasing holes 58 may be associated with a fibrous element-forming hole 56 such that the fluid exiting the fluid-releasing hole 58 is parallel or substantially parallel (rather than angled like a knife-edge die) to an exterior surface of a filament exiting the fibrous element-forming hole 56. In one example, the fluid exiting the fluid-releasing hole 58 contacts the exterior surface of a filament formed from a fibrous element-forming hole 56 at an angle of less than 30° and/or less than 20° and/or less than 10° and/or less than 5° and/or about 0°. One or more fluid releasing holes 58 may be arranged around a fibrous element-forming hole 56. In one example, one or more fluid-releasing holes 58 are associated with a single fibrous element-forming hole 56 such that the fluid exiting the one or more fluid releasing holes 58 contacts the exterior surface of a single filament formed from the single fibrous element-forming hole 56. In one example, the fluid-releasing hole 58 permits a fluid, such as a gas, for example air, to contact the exterior surface of a filament formed from a fibrous element-forming hole 56 rather than contacting an inner surface of a filament, such as what happens when a hollow filament is formed.

### Polymer Melt Composition

The polymer melt composition, for example an aqueous polymer melt composition such as an aqueous hydroxyl polymer melt composition, of the present invention comprises a melt processed fibrous element-forming polymer, such as a melt processed hydroxyl polymer, and a fast wetting surfactant according to the present invention.

The polymer melt compositions may already be formed or a melt processing step may need to be performed to convert a raw material fibrous element-forming polymer, such as a hydroxyl polymer, into a melt processed fibrous element-forming polymer, such as a melt processed hydroxyl polymer, thus producing the polymer melt composition. Any suitable melt processing step known in the art may be used to convert the raw material fibrous element-forming polymer into the melt processed fibrous element-forming polymer. “Melt processing” as used herein means any operation and/or process by which a polymer is softened to such a degree that it can be brought into a flowable state.

The polymer melt compositions may have a temperature of from about 50° C. to about 100° C. and/or from about 65° C. to about 95° C. and/or from about 70° C. to about 90° C. when spinning fibrous elements from the polymer melt compositions.

In one example, the polymer melt composition of the present invention may comprise from about 30% and/or from about 40% and/or from about 45% and/or from about 50% to about 75% and/or to about 80% and/or to about 85% and/or to about 90% and/or to about 95% and/or to about 99.5% by weight of the polymer melt composition of a fibrous element-forming polymer, such as a hydroxyl polymer. The fibrous element-forming polymer, such as a hydroxyl polymer, may have a weight average molecular weight greater than 100,000 g/mol

In one example, the fibrous elements and/or layered fibrous structure of the present invention produced via a polymer processing operation may be cured at a curing temperature of from about 110° C. to about 260° C. and/or from about 110° C. to about 230° C. and/or from about 120° C. to about 200° C. and/or from about 130° C. to about 185° C. for a time period of from about 0.01 and/or 1 and/or 5 and/or 15 seconds to about 60 minutes and/or from about 20 seconds to about 45 minutes and/or from about 30 seconds to about 30 minutes. Alternative curing methods may include radiation methods such as UV, e-beam, IR and other temperature-raising methods.

Further, the polymeric structure may also be cured at room temperature for days, either after curing at above room temperature or instead of curing at above room temperature.

The polymeric structures of the present invention may include melt spun fibers and/or spunbond fibers, staple fibers, hollow fibers, shaped fibers, such as multi-lobal fibers and multicomponent fibers, especially bicomponent fibers. The multicomponent fibers, especially bicomponent fibers, may be in a side-by-side, sheath-core, segmented pie, ribbon, islands-in-the-sea configuration, or any combination thereof. The sheath may be continuous or non-continuous around the core. The ratio of the weight of the sheath to the core can be from about 5:95 to about 95:5. The fibers of the present invention may have different geometries that include round, elliptical, star shaped, rectangular, and other various eccentricities.

One or more polymeric structures of the present invention may be incorporated into a multi-polymeric structure product, such as a layered fibrous structure and/or web, if the polymeric structures are in the form of fibers. Such a multi-polymeric structure product may ultimately be incorporated into a commercial product, such as a single- or multi-ply sanitary tissue product, such as facial tissue, bath tissue, paper towels and/or wipes, feminine care products, diapers, writing papers, cores, such as tissue cores, and other types of paper products.

### Non-Limiting Example of a Layered Fibrous Structure

### Example

### Layered Fibrous Structure Comprising Starch Filaments/Wood Pulp Fibers in a Single Ply Tissue Web

A polymer melt composition comprising 79% IPG starch commercially available from Ingredion Inc., 16% Ethylex 2035 (ethoxylated starch) commercially available from Tate & Lyle PLC, 0.6% Aerosol AOT—70PG (sulfosuccinate surfactant) available from Cytec Industries, Inc., 0.6% Hyperfloc NF301PWG (non-hydroxyl polymer), commercially available from Hychem Inc., 3.1% Urea glyoxal adduct crosslinking agent (dihydroxyethyleneurea) (containing less than 2% by weight of a base, for example triethanolamine), 0.003% Violet CT (hueing agent) commercially available from Milliken Chemical, and 0.7% Ammonium methane sulfonate (crosslinking facilitator). The polymer melt composition is cooked at approximately 125° C. and extruded from a co-rotating twin screw extruder at approx 50% solids (50% H₂O). The melt composition then passes through a heat exchanger to raise the temperature to approximately 175° C. The heated melt then passes to a flash extruder where water is flashed off and the melt cooled back to approximately 70° C.

The melt composition is then pumped to a meltblown spinnerette and attenuated with a 65° C. saturated air stream to form a nonwoven substrate having a basis weight of from about 10 g/m² to about 100 g/m². The filaments are then dried by convection drying before being deposited on a forming belt to form a filament web. These meltblown filaments are essentially continuous filaments.

Wood pulp fibers, Southern Softwood Kraft available as roll comminution pulp, is disintegrated by a hammermill and conveyed to an airlaid former via a blower. The wood pulp fibers are deposited onto the nonwoven substrate as a solid additive at a basis weight of from about 5 to 35 gsm.

The melt composition is also pumped to a second meltblown spinnerette and attenuated with a 65° C. saturated air stream to form a nonwoven substrate having a basis weight of from about 10 g/m² to about 100 g/m². The filaments are then dried by convection drying before being deposited on the web already in process as described above. These meltblown filaments are essentially continuous filaments.

The entire layered fibrous structure (sandwich) is then subjected to a thermal bonding process wherein the bond sites are formed with heat and pressure.

The bonded structure then undergoes a curing/crosslinking step by applying heat to the layered fibrous structure such that the layered fibrous structure reaches a temperature of about 200° C. for a sufficient time for sufficient crosslinking to occur.

The layered fibrous structure is then humidified to approximately 7-10 wt % moisture and rewound into a parent roll.

The single ply parent roll is then converted into a sanitary tissue product with perforations and an emboss pattern.

### Test Methods

Unless otherwise specified, all tests described herein including those described under the Definitions section and the following test methods are conducted on samples that have been conditioned in a conditioned room at a temperature of 23° C.±1.0° C. and a relative humidity of 50%±2% for a minimum of 24 hours prior to the test. All plastic and paper board packaging articles of manufacture, if any, must be carefully removed from the samples prior to testing. The samples tested are “usable units.” “Usable units” as used herein means sheets, flats from roll stock, pre-converted flats, layered fibrous structure, and/or single or multi-ply products. Except where noted all tests are conducted in such conditioned room, all tests are conducted under the same environmental conditions and in such conditioned room. Discard any damaged product. Do not test samples that have defects such as wrinkles, tears, holes, and like. All instruments are calibrated according to manufacturer's specifications.

### Basis Weight Test Method

Basis weight of a layered fibrous structure is measured on stacks of twelve usable units using a top loading analytical balance with a resolution of ±0.001 g. The balance is protected from air drafts and other disturbances using a draft shield. A precision cutting die, measuring 8.890 cm±0.00889 cm by 8.890 cm±0.00889 cm is used to prepare all samples.

With a precision cutting die, cut the samples into squares. Combine the cut squares to form a stack twelve samples thick. Measure the mass of the sample stack and record the result to the nearest 0.001 g.

The Basis Weight is calculated in g/m² as follows:

Basis Weight=(Mass of stack)/[(Area of 1 square in stack)×(No. of squares in stack)]Basis Weight(g/m²)=Mass of stack(g)/[79.032(cm²)/10,000(cm²/m²)×12]

Report result to the nearest 0.1 g/m². Sample dimensions can be changed or varied using a similar precision cutter as mentioned above, so as at least 645 square centimeters of sample area is in the stack.

### Average Diameter Test Method

A layered fibrous structure comprising filaments of appropriate basis weight (approximately 5 to 20 grams/square meter) is cut into a rectangular shape sample, approximately 20 mm by 35 mm. The sample is then coated using a SEM sputter coater (EMS Inc, PA, USA) with gold so as to make the filaments relatively opaque. Typical coating thickness is between 50 and 250 nm. The sample is then mounted between two standard microscope slides and compressed together using small binder clips. The sample is imaged using a 10× objective on an Olympus BHS microscope with the microscope light-collimating lens moved as far from the objective lens as possible. Images are captured using a Nikon D1 digital camera. A Glass microscope micrometer is used to calibrate the spatial distances of the images. The approximate resolution of the images is 1 μm/pixel. Images will typically show a distinct bimodal distribution in the intensity histogram corresponding to the filaments and the background. Camera adjustments or different basis weights are used to achieve an acceptable bimodal distribution. Typically 10 images per sample are taken and the image analysis results averaged.

The images are analyzed in a similar manner to that described by B. Pourdeyhimi, R. and R. Dent in “Measuring fiber diameter distribution in nonwovens” (Textile Res. J. 69(4) 233-236, 1999). Digital images are analyzed by computer using the MATLAB (Version. 6.1) and the MATLAB Image Processing Tool Box (Version 3.) The image is first converted into a grayscale. The image is then binarized into black and white pixels using a threshold value that minimizes the intraclass variance of the thresholded black and white pixels. Once the image has been binarized, the image is skeltonized to locate the center of each fiber in the image. The distance transform of the binarized image is also computed. The scalar product of the skeltonized image and the distance map provides an image whose pixel intensity is either zero or the radius of the fiber at that location. Pixels within one radius of the junction between two overlapping fibers are not counted if the distance they represent is smaller than the radius of the junction. The remaining pixels are then used to compute a length-weighted histogram of filament diameters contained in the image.

### Lint/Pilling Test Method

i. Sample Preparation—

Sample strips (a total of 4 if testing both sides, 2 if testing a single side) of layered fibrous structures and/or sanitary tissue products, which do not have abraded portions) 11.43 cm (4.5 inch) wide×30.48 cm to 40.64 cm (12-16 inch) long such that each sample strip can be folded upon itself to form a 11.43 cm (4.5 inch) wide (CD) by 10.16 cm (4.0 inch) long (MD) rectangular implement having a total basis weight of between 140 to 200 g/m² are obtained and conditioned according to Tappi Method #T4020M-88. For both side testing, makeup two rectangular implements as described above with a first side out and then two rectangular implements with the other side out (keep track of which are which).

For sanitary tissue products formed from multiple plies of layered fibrous structure, this test can be used to make a lint measurement on the multi-ply sanitary tissue product, or, if the plies can be separated without damaging the sanitary tissue product, a measurement can be taken on the individual plies making up the sanitary tissue product. If a given sample differs from surface to surface, it is necessary to test both surfaces and average the scores in order to arrive at a composite lint score. In some cases, sanitary tissue products are made from multiple-plies of layered fibrous structures such that the facing-out surfaces are identical, in which case it is only necessary to test one surface.

Each sample is folded upon itself to make a 4.5″ CD×4″ MD sample. For two-surface testing, make up 3 (4.5″ CD×4″ MD) samples with a first surface “out” and 3 (4.5″ CD×4″ MD) samples with the second surface “out”. Keep track of which samples are first surface “out” and which are second surface “out”.

For a dry lint/pilling test, obtain a 30″×40″ piece of Crescent #300 cardboard from Cordage Inc. (800 E. Ross Road, Cincinnati, Ohio, 45217). Using a paper cutter, cut out six pieces of cardboard of dimensions of 6.35 cm×15.24 cm (2.5 inch×6 inch). Puncture two holes into each of the six pieces of cardboard by forcing the cardboard onto the hold down pins of the Sutherland Rub tester. Center and carefully place each of the cardboard pieces on top of the previously folded samples with the tested side exposed outward. Make sure the 15.24 cm (6 inch) dimension of the cardboard is running parallel to the machine direction (MD) of each of the folded samples. Fold one edge of the exposed portion of the sample onto the back of the cardboard. Secure this edge to the cardboard with adhesive tape obtained from 3M Inc. (¾″ wide Scotch Brand, St. Paul, Minn.). Carefully grasp the other over-hanging tissue edge and snugly fold it over onto the back of the cardboard. While maintaining a snug fit of the sample onto the cardboard, tape this second edge to the back of the cardboard. Repeat this procedure for each sample. Turn over each sample and tape the cross direction edges of the sample to the cardboard. One half of the adhesive tape should contact the sample while the other half is adhering to the cardboard. Repeat this procedure for each of the samples. If the sample breaks, tears, or becomes frayed at any time during the course of this sample preparation procedure, discard and make up a new sample with a sample strip.

For a wet lint/pilling test, first prepare the testing surface by securely fastening a smooth surface foam pad (⅛″ thick, Poron quick Recovery Foam, adhesive back, firmness rating 13), having a length greater than or equal to 15.24 cm (6 inch) and a width greater than or equal to 12.70 cm (5 inch), to a flat and level table surface, positioned in such a way that its ≧12.70 cm length direction is parallel to the table edge, and is flush with the table edge. On top of this foam surface, adhere a piece of fine grade sandpaper (12.70 cm×15.24 cm, using double-sided tape or glue), with its shorter axis parallel to the table edge, and centered with respect to other dimensions of the foam. Position the folded sample such that one of its CD-axis sides is 0-¼ inch from the from the table surface and foam/sandpaper edge. Adhere the opposite edge of the sample (using ≧8″ length of Scotch brand ¾ inch transparent tape) with tape extending long enough to adhere to both sides of the table.

ii. Felt and Weight Component Preparation—

Cut a piece of a black test felt (F-55 or equivalent from New England Gasket, 550 Broad Street, Bristol, Conn. 06010) to the dimensions of 2¼″×7¼″. The felt is to be used in association with a weight. The weight may include a clamping device to attach the felt/cardboard combination to the weight. The weight and any clamping device total five (5) pounds. The weight is available from Danilee Company, San Antonio, Tex., and is associated with the Sutherland Rub Tester. The weight has a 2″×4″ piece of smooth surface foam attached to its contact face (⅛″ thick, Poron quick Recovery Foam, adhesive back, firmness rating 13). For the dry test, the felt is clamped directly against this foam surface, providing an effective contact area of 8 in² and a contact pressure of about 0.625 psi. For the wet test, an additional 1″×4″ foam strip (same foam as described above) is attached and centered in the length direction on top the 2″×4″ foam strip, thus, after clamping the felt against this surface, an effective contact area of 4 in² and a contact pressure of about 1.25 psi is established. Also, for the wet test only, after clamping the felt to weight apparatus, two strips of tape (4¼″-5¼″ in length, Scotch brand ¾″ width) are placed along each edge of the felt (parallel to the long side of the felt) on the felt side that will be contacting the sample. The untaped felt between the two tape strips has a width between 18-21 mm. Three marks are placed on one of the strips of tape at 0, 4 and 10 centimeters along the flat, test region of the test felt.

iii. Conducting Dry Lint/Pills Test—

The amount of dry lint and/or dry pills generated from a fibrous product according to the present invention is determined with a Sutherland Rub Tester (available from Danilee Company, San Antonio, Tex.). This tester uses a motor to rub a felt/weight component 5 times (back and forth) over the fibrous product, while the fibrous product is restrained in a stationary position.

First, turn on the Sutherland Rub Tester pressing the “reset” button. Set the tester to run 5 strokes at the lower of the two speeds. One stroke is a single and complete forward and reverse motion of the weight. The end of the rubbing block should be in the position closest to the operator at the beginning and at the end of each test.

Place the sample/cardboard combination on the base plate of the tester by slipping the holes in the board over the hold-down pins. The hold-down pins prevent the sample from moving during the test. Hook the felt/weight combination into the tester arm of the Sutherland Rub Tester, and gently place it on top of the sample/cardboard combination. The felt must rest level on the calibration sample and must be in 100% contact with the calibration sample surface (use a bubble level indicator to verify). Activate the Sutherland Rub Tester by pressing the “start” button.

Keep a count of the number of strokes and observe and make a mental note of the starting and stopping position of the felt covered weight in relationship to the sample. If the total number of strokes is five and if the position of the calibration felt covered weight is the same at the end as it was in the beginning of the test, the test was successful performed. If the total number of strokes is not five or if the start and end positions of the felt covered weight are different, then the instrument may require servicing and/or recalibration.

Once the instrument is finished moving, remove the felt covered weight from the holding arm of the instrument, and unclamp the felt from the weight. Lay the test felt on a clean, flat surface.

iv. Conducting Wet Lint/Pills Test—

Wet lint/pills are determined by pulling, during one pass, a partially wetted felt/weight component over a sample.

To wet the felt, pipette 0.6 ml of deionized water on to the felt, between the 0 and 4 cm marks, as represented on the tape attached to the felt. Before the water soaks into the felt, use a metal ruler with a width of ¾″, to spread the water uniformly across the 0-4 cm marked wet zone without spilling onto the tape or into the dry zone (between the 4 and 10 cm marks).

After the water is uniformly distributed and fully penetrated into the felt (not beaded up at all), place weight-felt apparatus on the sample such that the felt wetted region is ≦¼″ from the edge of sample and tape. After approximately one second, pull the knob horizontally until the apparatus is completely off the table—the pulling process should take 0.5 to 1.5 seconds. Pull the weight in a manner to avoid placing any additional force on the felt/weight component other than the horizontal pull force. The pulling process should occur as a substantially continuous or continuous motion. Record if sample sheet tears significantly due to felt rubbing, and/or if pieces fall off (onto floor) during the test.

Carefully remove the felt from the weight, store in a safe, flat place, and allow to dry before imaging (≧24 hours, standard conditions). Do not stack multiple layers of felt on top one another to prevent sticking and lint/pill transfer.

The next step is to complete image capture, analysis, and calculations on the test felts as described below.

vi. Image Capture—

The images of the felt (untested), sample (untested) and felt (tested) are captured using a computer and scanner (Microtek ArtixScan 1800f). Be certain that scanner glass is clear and clean. Place felts centered on scanner, face down. Adjust image capture boundaries so that all felts are included into the captured image. Set-up the scanner to 600 dpi, RGB, and 100% image size (no scaling). After successfully imaging the felts, save the image as an 8-bit RGB TIFF image, remove felts from scanner, and repeat from process until all felts images are captured.

Additional images of the sample (untested) may need to be captured (in the same manner) if they have an average luminance (using Optimas software) significantly less than 254 (less than 244), after being converted to an 8-bit gray-scale image. Also, an image of a known length standard (e.g., a ruler) is taken (exposure difference does not matter for this image). This image is used to calibrate the image analysis software distance scale.

vii. Image Analysis—

The images captured are analyzed using Optimas 6.5 Image

Analysis software commercially available from Media Cybernetics, L.P. Imaging set-up parameters, as listed herein, must be strictly adhered to in order to have meaningfully comparative lint score and pill score results.

First, an image with a known length standard (e.g., a ruler) is brought up in Optimas, and used to calibrate length units (millimeters in this case). For dry testing, the region of interest (ROI) area is approximately 4500 mm2 (90 mm by 50 mm), and the wetted and dragged ROI area is approximately 1500 mm2 (94 mm by 16 mm). The exact ROI area is measured and recorded (variable name: ROI area). The average gray value of the unrubbed region of the test felt is used as the baseline, and is recorded for determining the threshold and lint values (variable name: untested felt GV avg). It is determined by creating a region of interest box (ROI) with dimensions approximately 5 mm by 25 mm on the untested, unrubbed area of the black felt, on opposite ends of the rubbed region. The average of these two average gray value luminaces for each of the ROI's is used as the untested felt GV average value for that particular test felt. This is repeated for all test felts analyzed. The test sheet luminance is typically near saturated white (gray value 254) and fairly constant for samples of interest. If believed to be different, measure the test sheet in a similar fashion as was done for the untested felt, and record (variable name=untested sheet GV avg). The luminance threshold is calculated based on the untested felt GV avg and untested sheet GV avg as follows:

For the dry lint/pilling test felts:

(untested_sheet_GV_avg−untested_felt_GV_avg)*0.4+untested_felt_GV_avg

For the wet lint/pilling test felts:

(untested_sheet_GV_avg−untested_felt_GV_avg)*0.25+untested_felt_GV_avg

The test felt image is opened, and the ROI and its boundaries are created and properly positioned to encompass a region that completely contains pills and contains the highest concentration of pills on the rubbed section of the test felt. The average luminance for the ROI is recorded (variable name: ROI GV avg). Pills are determined as follows: Optimas creates boundary lines in the image where pixel luminance values cross through the threshold value (e.g., if the threshold is 120, boundary lines are created where pixels of higher and lower value exist on either side. The criteria for determining a pill is that it must have an average luminance greater than the threshold value, and have a perimeter length greater than 0.5 mm. The sum of the pilled areas variable name is: Total Pilled Area.

Measurement data of the ROI, and for each pill is exported from Optimas to a spreadsheet for performing the following calculations.

viii. Calculations—

The data obtained from the image analysis is used in the following calculations:

Pilled Area %=Percent of area covered by pilling=Total Pilled Area/ROI area

Lint Score=Gray value difference between unpilled area of the rubbed test felt area and the untested felt

Lint Score=unpilled felt Gray Value avg−untested felt Gray Value avg

where: unpilled felt Gray Value avg=[(ROI Gray Value avg*ROI area)−(pilled Gray Value avg*pilled area)]/Total Unpilled Area

By taking the average of the lint score of the first-side surface and the second-side surface, the lint is obtained which is applicable to that particular web or product. In other words, to calculate lint score, the following formula is used:

$$Dry Lint Score = \frac{\begin{matrix}Dry Lint Score , {1}^{st} side + \\ Dry Lint Score , {2}^{nd} side\end{matrix}}{2}$$

$$Dry Pill Area \% = \frac{\begin{matrix}Dry Pill Area \% , {1}^{st} side + \\ Dry Pill Area \% , {2}^{nd} side\end{matrix}}{2}$$

$$Wet Lint Score = \frac{\begin{matrix}Wet Lint Score , {1}^{st} side + \\ Wet Lint Score , {2}^{nd} side\end{matrix}}{2}$$

$$Wet Pill Area \% = \frac{\begin{matrix}Wet Pill Area \% , {1}^{st} side + \\ Wet Pill Area \% , {2}^{nd} side\end{matrix}}{2}$$

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A layered fibrous structure comprising a first outer layer comprising a plurality of hydroxyl polymer filaments present in the first outer layer at a basis weight of at least 10 gsm.

2. The layered fibrous structure according to claim 1 wherein the hydroxyl polymer filaments are present in the first outer layer at a basis weight of at least 12 gsm.

3. The layered fibrous structure according to claim 1 wherein the hydroxyl polymer filaments are present in the first outer layer at a basis weight of at least 10 to about 30 gsm.

4. The layered fibrous structure according to claim 1 wherein the layered fibrous structure further comprises a second layer comprising a plurality of fibers.

5. The layered fibrous structure according to claim 4 wherein the second layer fibers comprise pulp fibers.

6. The layered fibrous structure according to claim 4 wherein the fibers are present in the second layer at a basis weight of at least 6 gsm.

7. The layered fibrous structure according to claim 4 wherein the fibers are present in the second layer at a basis weight of at least 6 gsm to about 50 gsm.

8. The layered fibrous structure according to claim 4 wherein the second layer is a coform layer comprising commingled fibers and filaments.

9. The layered fibrous structure according to claim 8 wherein the coform layer exhibits a basis weight of at least 6 gsm.

10. The layered fibrous structure according to claim 8 wherein the coform layer exhibits a basis weight of at least 6 gsm to about 50 gsm.

11. The layered fibrous structure according to claim 1 wherein the hydroxyl polymer filaments comprise a hydroxyl polymer selected from the group consisting of: starch, starch derivatives, starch copolymers, chitosan, chitosan derivatives, chitosan copolymers, cellulose, cellulose derivatives, cellulose derivatives, cellulose copolymers, hemicelluloses, hemicelluloses derivatives, hemicelluloses copolymers, and mixtures thereof.

12. The layered fibrous structure according to claim 1 wherein the hydroxyl polymer filaments comprise a hydroxyl polymer selected from the group consisting of: starch, starch derivatives, and mixtures thereof.

13. A single- or multi-ply sanitary tissue product comprising a layered fibrous structure according to claim 1.

14. The sanitary tissue product according to claim 13 wherein the sanitary tissue product is in roll form.
